(19) United States
(12) Patent Application Publication
(10) Pub. No.: US 2022/0375539 A1
(43) Pub. Date: Nov. 24, 2022

(54) SYSTEMS AND METHODS FOR PREDICTING PROTEINS

(71) Applicant: GEAENZYMES CO., Sunnyvale, CA (US)

(72) Inventors: Leonardo Alvarez, Santiago (CL); Roberto Ibañez, Santiago (CL); Patricio Alegre, Santiago (CL); Pedro Retamal, Santiago (CL); Simon Correa, Santiago (CL); Romualdo Paz, Santiago (CL); Javier Caceres-Delpiano, Santiago (CL); Cynthia Sanhueza, Santiago (CL); Juan Cristobal Jimenez, Santiago (CL)

(21) Appl. No.: 17/753,024

(22) PCT Filed: Aug. 21, 2020
(86) PCT No.: PCT/US20/47374

§ 371 Date: Feb. 15, 2022

Related U.S. Application Data
(60) Provisional application No. 62/891,202, filed on Aug. 23, 2019.

(51) Int. Cl. G16B 15/20; G16B 40/00; C07K 1/00

(52) U.S. Cl. CPC G16B 15/20; G16B 40/00; C07K 1/00

(57) ABSTRACT

Embodiments of the invention include systems and methods that enable the identification of candidate proteins that have desired features of a target protein. An example method comprises receiving first and second input proteins. The method further comprises applying a first machine learning model to the first and second input proteins to generate corresponding fragments. The method further comprises applying a second machine learning model to the fragments, wherein applying the second machine learning model comprises generating an encoded representation in a multidimensional space for each of the fragments. The method also comprises generating a similarity score between the fragments from the first input and the second input. The method then comprises generating a hierarchical scale of similarity between the first and second inputs according to the similarity score and selecting candidate proteins based on the hierarchical scale.

### BACKGROUND

### Field

This disclosure relates generally to computational systems and methods, and more particularly to systems and methods for predicting alternative functional proteins, for example animal-free or plant-based functional proteins based on a target animal interest protein.

### Description of Related Art

Proteins are widely used as ingredients within various industries given their physical-chemical properties, for example the food and cosmetic industries.

In recent years, consumer demand for animal-free proteins has prompted an increase in the development of systems and methods for identifying and/or predicting animal-free or plant-based functional proteins. However, existing systems and methods for predicting such functional proteins are expensive, time consuming, and not necessarily accurate.

Accordingly, current systems and methods may be insufficient for identifying or predicting animal-free functional proteins. For example, some current systems and methods use prediction models that do not reflect complexities of protein structures, do not use defined or identified relationships of a known protein to predict evolutionary terms for the protein, or do not use comparisons of proteins in multidimensional space to identify degrees of similarity between proteins. Currently, a protein sequence is the most widely-used predictor of protein function and, thus, is used in many current systems and methods.

### SUMMARY

In accordance with an aspect, there is provided a system for predicting an alternative functional protein. The system implemented by one or more computers includes: a sub-system, apparatus, or module to receive a plurality of inputs, where each input is a protein sequence and where one or more is an interest protein and the rest of the inputs are substitute candidate proteins. The system further includes a sub-system, apparatus, or module to process each of the plurality of substitute candidate proteins inputs with a computational algorithm to split the substitute candidate protein into fragments of interest. The system further comprises a sub-system, apparatus, or module to process each of the plurality of inputs (both interest protein and the substitute candidate proteins) with an artificial intelligence model to get an encoded representation of the proteins in a multidimensional space. The system further comprises a sub-system, apparatus, or module to process each of the plurality of inputs to generate a similarity score between the interest inputs and the substitute candidate proteins. The system also comprises a sub-system, apparatus, or module to generate a hierarchical scale of similarity between interest proteins and substitute candidate proteins and to select the higher scored or the more similar substitute candidate proteins for each of the interest protein inputs.

In accordance with an aspect, there is provided a system for predicting an alternative functional protein. The system implemented by one or more computers includes: a sub-system, apparatus, or module to receive a plurality of inputs, where each input is a protein sequence and where one or more is an interest protein and the rest of the inputs are substitute candidate proteins. The system further includes a sub-system, apparatus, or module to process each of the plurality of substitute candidate proteins inputs with a computational algorithm to split the substitute candidate protein into fragments of interest. The system further comprises a sub-system, apparatus, or module to process each of the plurality of inputs (both interest protein and the substitute candidate proteins) with an artificial intelligence model to get an encoded representation of the proteins in a multidimensional space. The system further comprises a sub-system, apparatus, or module to process each of the plurality of inputs to generate a similarity score between the interest inputs and the substitute candidate proteins. The system also comprises a sub-system, apparatus, or module to generate a hierarchical scale of similarity between interest proteins and substitute candidate proteins and to select the higher scored or the more similar substitute candidate proteins for each of the interest protein inputs.

In another aspect, there is provided a method of predicting an alternative functional protein. The method comprises receiving a first input comprising a target protein sequence having a feature of interest and receiving a second input comprising a candidate protein. The method further comprises applying a first machine learning model to the target protein sequence to generate fragments of interest from the target protein sequence and applying the first machine learning model to the candidate protein to generate fragments from the candidate protein that corresponds to the fragments of interest from the target protein sequence. The method also comprises applying a second machine learning model to the fragments of interest and the fragments from the candidate protein, wherein applying the second machine learning model comprises generating an encoded representation in a multidimensional space for each of the fragments of interest and the fragments from the candidate protein. The method additionally comprises generating a similarity score between the target protein sequence and the candidate protein based on a similarity between the fragments of interest from the target protein sequence and the fragments from the candidate protein and generating a hierarchical scale of similarity between the target protein sequence and a plurality of candidate proteins comprising the candidate protein according to the feature of interest and the similarity score. The method also comprises selecting candidate proteins from the plurality of candidate proteins based on the hierarchical scale, wherein higher scores on the hierarchical scale indicate candidate proteins or the more similar substitute candidate proteins inputs for each of the interest inputs.

In the above corresponding aspect, the first machine learning model is configured to generate the fragments based on splitting the target protein sequence and the candidate protein into functional domain fragments. In any of the above corresponding aspects, the second machine learning module comprises a plurality of fully connected layers. In any of the above corresponding aspects, the second machine learning module comprises a plurality of convolutional neural network layers. In any of the above corresponding aspects, the second machine learning module comprises a plurality of recurrent neural network layers configured to identify the beginning and end of functional domains. In any of the above corresponding aspects, the layers are connected with direct connections or residual neural networks connections. In any of the above corresponding aspects, the computational algorithm to split comprises a module to allow the input to be divided into a defined size. In any of the above corresponding aspects, the first machine learning model is configured to generate the fragments of interest and the fragments of the candidate protein to have different sizes or functional domains. In any of the above corresponding aspects, applying the second machine learning model comprises encoding an amino-acid representation of the one or more candidate proteins in a multidimensional space given a local context of the candidate proteins. In any of the above corresponding aspects, encoding the amino-acid representation comprises applying a sub-model having a plurality of fully-connected layers, a plurality of convolutional neural networks layers, or a plurality of recurrent neural networks layer to compress the amino-acid representation given the local context of the candidate proteins. In any of the above corresponding aspects, the layers are connected with direct connections or residual neural networks connections. In any of the above corresponding aspects, further comprising training the sub-model to predict a probability of the amino acid beginning or ending at a certain position in the candidate protein given the local context. In the above corresponding aspects, further comprising training the sub-model using all known or predicted protein sequences. In any of the above corresponding aspects, the second machine learning model comprises a sub-model to encode the candidate proteins and the target protein in a multidimensional space given a protein sequence and a compress positional representation information. In the above corresponding aspect, the sub-model comprise a plurality of fully-connected layers, a plurality of convolutional neural networks layers, or a plurality of recurrent neural networks layer to compress the amino-acid representation given the local context of the candidate proteins. In the above corresponding aspect, the layers are connected with direct connections or residual neural networks connections. In the above corresponding aspect, further comprising training the sub-model to predict a protein structure, and a function of protein, and a contact-map of protein. In any of the above corresponding aspects, generating a similarity score between the target protein sequence and each candidate protein comprises applying a third machine learning model to generate the similarity score between the target protein and the one or more candidate proteins. In the above corresponding aspect, the third machine learning model comprises a plurality of fully connected layers, a plurality of convolutional neural networks layers, or a plurality of recurrent neural networks layer to compare two multidimensional protein representations. In the above corresponding aspect, the similarity score is a hierarchical number that contains information of the protein sequence, structure and function. In the above corresponding aspect, further comprising training the third machine learning to predict similarity of proteins using the SCOP hierarchical information. In the above corresponding aspect, where the module ranker orders the candidates from the best rated to the worst.

In another aspect, there is provided a method of predicting an alternative functional protein using a computational system implemented by one or more computers. The method comprises receiving a plurality of inputs, each input is a protein sequence where one or more are animal protein and the rest of the inputs are plant-based, animal-free candidate proteins. The method further comprises processing each of the plurality of plant-based, animal-free candidate proteins inputs with a computational algorithm to split the protein in fragments of interest and processing each of the plurality of inputs with an artificial intelligence model to get an encoded representation in a multidimensional space. The method also comprises processing each of the plurality of inputs to generate a similarity score between the animal protein inputs and the plant-based, animal-free candidate proteins. The method additionally comprises generating a hierarchical scale of similarity between animal protein inputs and plant-based, animal-free candidate proteins. The method also comprises selecting the higher scores or the more similar substitute candidate proteins inputs for each of the interest inputs.

In any of the above corresponding aspects, the plant-based, animal-free candidate proteins are split into functional domains and/or into a defined size. In any of the above corresponding aspects, the plant-based, animal-free candidate proteins and the animal-origin protein are encoded using a trained artificial intelligence model, given an encoded representation of proteins and/or fragments. In any of the above corresponding aspects, the plant-based, animal-free candidate proteins previously encoded in a multidimensional space are compared using a hierarchical similarity metric. In any of the above corresponding aspects, the plant-based, animal-free candidate proteins previously compared using a hierarchical similarity metric are ranked from the best rated to the worst. In any of the above corresponding aspects, the best plant-based, animal-free candidate proteins are selected given a number of max candidates or if the score exceeds a threshold. In any of the above corresponding aspects, the best selected plant-based, animal-free candidate proteins are bioinformatically simulated and/or synthesized in the laboratory to verify the activity and fulfill the desired function.

In another aspect, there is provided a system comprising a processor and a memory including instruction to program the processor to perform the method of any of the aspects described above.

In another aspect, there is provided any of the methods described above, wherein the at least one fragment of interest is a target antifungal protein. In another aspects, there is provided any of the methods described above, further comprising identifying, based on substitute candidate proteins, alternative antifungal proteins to a target antifungal protein, wherein the at least one fragment of interest comprises a feature of the target antifungal protein. In another aspect, there is provided any of the methods described above, further comprising identifying, based on substitute candidate proteins, alternative enzymes to a target enzyme, wherein the at least one fragment of interest comprises a feature of the target enzyme.

### BRIEF DESCRIPTION OF THE DRAWINGS

The aspects described herein, as well as other features, aspects, and advantages of the present technology will now be described in connection with various implementations, with reference to the accompanying drawings. The illustrated implementations, however, are merely examples and are not intended to be limiting. Throughout the drawings, similar symbols typically identify similar components, unless context dictates otherwise. Note that the relative dimensions of the following figures may not be drawn to scale.

FIG. 1 is a block diagram of a system configured to predict or identify an alternative protein.

FIG. 2 is a block diagram of a module of the system of FIG. 1 that is configured to split or fragment proteins.

FIG. 3 is a block diagram of a module of the system of FIG. 1 that is configured to encode protein information in a numerical codification.

FIG. 4 is a block diagram of a module of the system of FIG. 1 that is configured to compare and hierarchically rank protein information using a numerical coding.

FIG. 5 is a first view of a pairing of protein structures including an animal-based protein and a corresponding plant-based protein as generated by the system of FIG. 1 based on a crystal and computation prediction.

FIG. 6 is a second view of the pairing of protein structures of FIG. 5.

FIG. 7 shows an alignment of the two protein sequences of FIGS. 5 and 6 that includes a protein of animal origin and a corresponding, animal-free protein generated by the system of FIG. 1.

FIG. 8 shows a table of results of a comparison of the animal-based protein with the animal-free protein of FIGS. 5, 6, and 7.

FIG. 9 shows a plot of comparisons between the animal-based and corresponding animal-free protein structures as stored in a protein data bank.

FIG. 10A is a block diagram corresponding to an aspect of a hardware and/or software component of an example embodiment of a device that implements the system of FIG. 1.

FIG. 10B illustrates one possible organization of a networked computing system that can dynamically generate proteins based on target protein features using machine learning models, in accordance with an exemplary embodiment.

FIG. 11 shows a representation of training of the models implemented by the system of FIG. 1.

FIG. 12 shows a representation of applying the models implemented by the system of FIG. 1.

FIGS. 13A-13D show a representation of design of the de novo protein variants using the system 1 of FIG. 1. (FIG. 13A).

FIG. 14 shows representations of RMSD and relative binding energies for AC2-WT.

FIGS. 15A and 15B show representations of contact analyses for AC2-WT.

FIG. 16 shows representations of RMSD and relative binding energies for DNv1-AC2.

FIGS. 17A and 17B shows representations of contact analyses for DNv1-AC2.

FIG. 18 shows representations of RMSD and relative binding energies for DNv2-AC2.

FIGS. 19A and 19B shows representations contact analyses for DNv2-AC2.

FIGS. 20A and 20B shows a representation of purification and activity of WT and de novo AC2 peptides fused to MBP protein.

FIGS. 21A and 21B shows a representation of Agar dilution assay comparing the antifungal activity of DNv1-AC2-MBP and DNv2-AC2-MBP against A. niger.

### DETAILED DESCRIPTION

In the following detailed description, reference is made to the accompanying drawings, which form a part thereof. In the drawings, similar symbols typically identify similar components, unless context dictates otherwise. Thus, in some embodiments, part numbers may be used for similar components in multiple figures, or part numbers may vary depending from figure to figure. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and made part of this disclosure.

Proteins are widely used as ingredients for products in various industries (for example, the food and cosmetic industries) because of the physical-chemical properties of the proteins. For example, egg whites are often used in confections and confectionery applications due to foam forming properties of the protein albumin that is present in the egg white. Gelatin is another example of an animal protein that is widely used in food applications due to its gelling properties.

Recently, demand has grown for alternatives to animal-based proteins. However, existing systems and methods of obtaining animal-free functional proteins are expensive and time consuming or not be able to identify alternatives based on specified features of the animal-based proteins.

Accurately predicting a protein's function can be a fundamental tool in the discovery of new medicines, food ingredients, cosmetics and treatments. However, the number of factors governing function is extensive and relationships between protein functions and other aspects are poorly understood, making such protein prediction difficult. Furthermore, the accuracy of the prediction may be dependent on the factors being considered by a predictive model used to make the prediction of the protein's function. Protein structures, and their effects on or interactions with environmental factors, are complex. For example, a given interaction between a substrate and the protein can be affected by a distance, angle, atom type, charge and polarization, and surrounding stabilizing or destabilizing environmental factors.

In some embodiments, knowledge-based scoring functions implemented by systems and methods that use protein sequence information can predict a protein's function or features of the protein, such as stability, solubility and activity. For example, an algorithm applied by the knowledge-based scoring functions can predict evolutionary terms for a protein based on defined relationships with a set of annotated proteins or protein functions such as protein stability, solubility, and activity. Alternatively, or additionally, the systems and methods may integrate machine learning to enable prediction of structure and functions of proteins based on the defined relationships with the set of annotated proteins. For example, a protein structure (for example, of a target protein) can be used directly or indirectly to predict a structure and/or function of another protein.

The present disclosure provides systems and methods to search for and/or identify candidate replacement proteins for interest proteins or desired proteins (hereinafter referred to as one or more of “target”, “interest”, and/or “desired” proteins). A plurality of interest proteins may represent an amino acid sequence that has a specific structural or enzymatic function. This function may be given or influenced by one or more of an amino acid composition, a three-dimensional structure, or an interaction with a particular environment. Using any of these characteristics of interest, a search may be performed to find similar characteristics in a determined group of proteins, grouped based on other characteristics, like sequence size, or given that the proteins are coded in the genetic material of an organism, or other properties. Details of predicting one or more proteins based on a desired or target protein function of interest (e.g., based on the composition, structure, or interaction) are provided below with reference to the figures and corresponding description.

In some embodiments, a user may wish to identify the replacement candidate protein from a plurality of such proteins that has a similar structural or enzymatic function as the interest protein. In some embodiments, the systems and methods described herein may receive the protein of interest and identify a specific fragment or fragments related to the desired protein structure. Alternatively, or additionally, the systems and methods may receive the protein of interest and assume that the entire protein sequence is of interest. For example, when identifying the protein of interest, the system may determine that only a portion of the protein of interest relates to the desired feature. For example, the systems and methods may incorporate a loop to gradually delete the ends of the entered interest protein sequence (left and right ends) so as to obtain the minimum necessary sequence of the interest protein related to the desired feature. Alternatively, the systems and methods described herein need not reduce the interest and candidate protein sequences because representation strategies, as described herein, can be used to address “excess” sequence information between the interest and candidate proteins and underweight the importance of the corresponding amino acids.

Once the systems and methods identify, from the plurality of candidate proteins, the candidate protein(s) of the plurality option is/are identified, the systems and methods may perform computational simulations to verify the stability and interaction properties of the identified candidate protein(s) under the desired conditions. The candidate protein(s) that pass these simulations (i.e., have appropriate stability and interaction properties) may then be experimentally tested in the laboratory.

The systems and methods described herein may receive, as inputs, the target protein and the candidate protein of the plurality of candidate proteins. A first module of the system may parse, as part of the methods described herein, the candidate substitute protein input into the system into fragments of the candidate substitute proteins that include the one or more desired features of the target protein. The first module may return either one or more of the fragments that include the desired feature or the entire candidate substitute protein.

A second module may generate, using an initial language model, an interpretation of the fragments and/or the entire candidate substitute protein generated by the first module and/or the target protein. The interpretation may comprise an internal representation and concatenated. The second module predicts protein contact maps and secondary structures as two different tasks, for example using another two Bi-LSTM model stacked layers, each with 1,024 hidden dimensions and a projection, in addition to the initial language model of the second module. For the contact maps and secondary structure predictions, the system may cross-entropy as a loss function and train the models for five epochs using the SCOP database filtered at 40% identity, which includes more than 30,000 structures. Therefore, the second module may allow the system to capture structural information from the inputs. SCOP is a database with many proteins and information related to family, superfamily, fold, and so forth. As described herein, the models applied by the system 1 are trained to compare two sequences of proteins and predict the similarity between them.

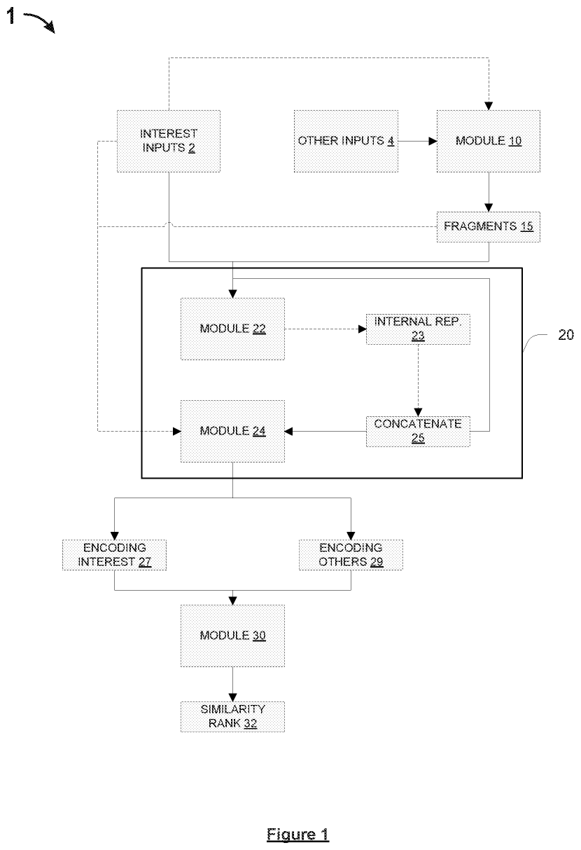

A third module compares the encoding interest representations from the second module (e.g., compares the target protein and the candidate protein with respect to the desired feature in the target protein) to generate a score indicative of how similar the candidate protein and the target protein are, relative to the feature of interest. Similarity rank is output and used by the system to determine which of the candidate proteins is best representative of the desired feature of the interest protein. The similarity rank is the threshold that let us decide if the suggested candidates will pass to experimental testing. So, the output will only work as a hierarchical organization of the best candidates to be tested in the laboratory. FIG. 1 is a block diagram of a system 1 configured to predict or identify an alternative functional protein of a plurality of candidate proteins when provided with a target protein or similar input having at least one desired feature. The system 1 may implement a method for identifying, from the plurality of candidate proteins, which one or more candidate proteins best match the target protein or best match a feature of the target protein. A user of the system 1 may wish to identify, from the plurality of candidate proteins, which candidate protein can be used as a substitute for the target protein. More specifically, the system 1 compares each candidate protein with the target protein to determine a similarity between a desired feature of the target protein and the candidate protein and generates a score for the candidate protein. The system 1 further ranks each of the plurality of candidate proteins to identify which candidate protein has the highest score. The candidate protein with the highest score of the plurality of candidate proteins may have the highest similarity with the desired feature of the target protein. The candidate protein with the highest score may then be used as a substitute of the target protein to obtain the desired feature.

The system 1 comprises a module 10, a module 20 comprising modules 22 and 24, internal representation block 23 and concatenate block 25, and a module 30. Each of these modules or blocks of the system 1 may be implemented by or comprise a software module or a hardware processing module. The modules and blocks of the system 1 may rank the candidate proteins (or fragments thereof) that share functions with the target or interest protein based on an analysis of the structure, interaction, or composition of the candidate proteins as compared to the target or interest protein.

The module 10 receives an interest protein (for example, the target protein) at interest inputs 2 and each candidate protein (for example, the candidate protein) at other inputs 4. Thus, the interest input 2 represents the target protein(s) or target fragment(s) that include one or more protein features that are of interest and are to be duplicated or replicated based on a substitute protein and the other inputs 4 represents the candidate substitute protein(s) that will be searched to identify features that correspond to (e.g., are similar to) the one or more protein features of interest in the target protein(s) or fragment(s).

The module 10 may control division (for example, by another module or component not shown) or fragmentation of the interest protein from the interest input 2 and/or the candidate protein from the other input 4 into protein fragments or chunks 15. Alternatively, or additionally, the module 10 may divide the interest protein and the candidate protein into the protein fragments or chunks 15 itself. For example, if the interest protein (or the candidate protein) comprises more than one functional domain, the module 10 may fragment the interest protein (or the candidate profile) into simple domains, which can be searched separately.

The protein fragments or chunks 15 may have different sizes and/or be defined by different search criteria, for example minimal structural domains, minimal identity domains, multiple defined sizes, and so forth. In some embodiments, the module 10 uses a sliding window to divide the interest and candidate proteins into a fragment set of predefined sizes of the protein fragments 15 (as established by the sliding window). The size of the window used to divide the interest and candidate proteins into the fragment set may be related to one of a feature or a structure of the target protein or the candidate protein, where the structure of a protein is related to its function. Protein structure (for example, of the interest protein and/or the candidate protein) may determine a function of the protein, and in some embodiments, similar protein structures can be related to similar protein functions so that comparisons of the structures of proteins can be used to compare functions of the proteins. Accordingly, a parameter such as the size of the window can enable searching of conserved structural similarities of different sizes. By collecting different structural fragments (for example, as generated by the module 10) the user can extrapolate the structure of an unknown protein.

As the sliding window moves throughout an amino acid sequence comprising, for example, the interest protein (from the interest inputs 2) and/or candidate protein (from the other inputs 4) within the search group, the sliding window may move with a predefined or user set stride between subsequent windows. The window size may be related to a target sequence length, for example according to a function such as windows_size=target_length+/−tolerance of approximately 10-15 amino acids. Thus, the stride may correspond to a jump between windows, and the stride may jump through the corresponding protein to enable comparison all the possible windows among the interest protein and the candidate protein, thereby making the comparison and scoring algorithm of FIG. 1 more efficient. In some embodiments, the stride size may depend on a size of the interest protein. For example, the stride size may comprise 20 amino acids for a first protein having a first protein size while the stride size may comprise 5 amino acids for a second protein having a second protein size that is smaller than the first protein size. The protein fragments 15 generated by the module 10 may share residues or sizes (e.g., fragment size and/or strides between windows), where the protein fragments 15 have a size corresponding to the predefined windows. In some embodiments, the protein fragments 15 generated by the module 10 may have different residues or sizes and/or strides between windows.

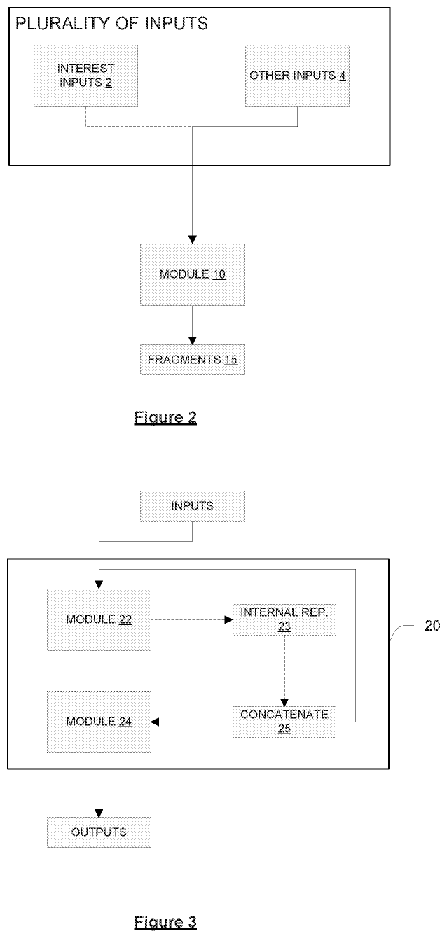

In some embodiments, the module 10 (as an individual module or as a combination of multiple modules, not shown) may apply one or more algorithms to process the sequences and divide the plurality of proteins (interest proteins from the interest inputs 2 and/or candidate proteins from the other inputs 4) by minimal structural or functional domains. In some embodiments, the one or more algorithms may include artificial intelligence algorithms, including but not limited to neural networks or recurrent neural networks stacked with direct connections and residual connections within layers. In some embodiments, the one or more algorithms implements one or more recurrent networks. Further details regarding the algorithms and processes of module 10 are provided below with respect to FIG. 2.

In some embodiments, the plurality of protein sequences and features of such protein sequences are compressed into a latent information space (for example, encoded into a multidimensional representation, as described in more detail below). The multidimensional representation is processed to identify/extract enough information to predict specific sites within one or more sequences of the plurality of sequences where protein domains/fragments start or end. In some embodiments, the module 10 uses one or more or a combination of neural or recurrent networks to predict protein fragment sizes based on the protein sequences and features of the protein sequences. In some embodiments, the module 10 may predict the protein fragment sizes based on specific functional domains that can be predicted using one or more pre-trained neural networks or similar systems. For example, the module 10 may employ an algorithm that enables the user (or the system 1) to set the size of the window described above to look for structural fragments of different sizes. In some embodiments, the algorithms or models applied by the module 10 are trained and applied to the candidate or interest protein to split the protein having multiple structural domains into fragments (for example, corresponding to the multiple structural domains) with a tolerance of +/−20 amino acids where the protein has more than one functional domain. Alternatively, or additionally selectively, the window size for fragmenting the proteins into fragments uses a window size established by the relationship windows_size=target_length+/−10 amino acids, as identified above.

In some embodiments, the input (e.g., the other inputs 4) for the module 10 comprises a plurality of numerical representations of the amino acid sequence of potential substitute proteins, keeping or maintaining the sequential nature of proteins. In some embodiments, the module 10 transforms the amino acid sequence into a numerical representation. This encoding or transformation of protein fragments (i.e., the amino acid sequence) into the numerical representation can be done in any number of ways. In a first technique, the module 10 applies one or more language models trained using a next token prediction (where the next token is a next amino acid for purposes of prediction). Alternatively, or selectively, the language models can be trained by masking some amino acids in the protein sequence and predict it.

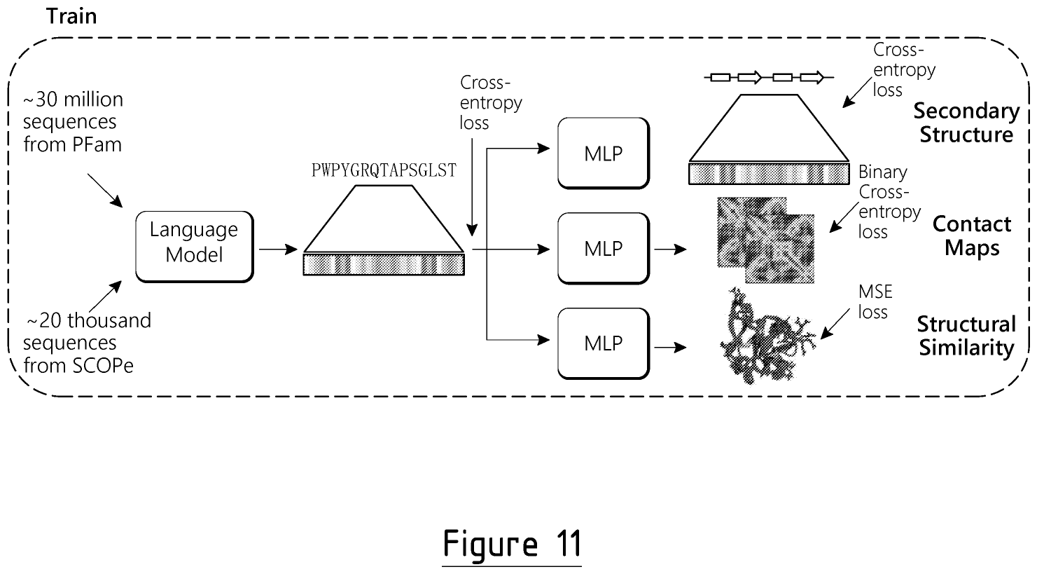

The transformation into the numerical representation can occur before the proteins are fragmented at the module 10 in manner that does not impact the sliding window fragmentation, which will then fragment based on the numerical representation. In some embodiments, selection between the next token and mask token prediction may be based on a size of the interest and/or candidate protein, where next token prediction training may be used for sequences of more than 512 amino acids and either the next token prediction or the mask token prediction can be selected for sequences of less than 512 amino acids. An example of the language model used to encode the protein sequences may be the BiLSTM language model, discussed in further detail below with reference to FIGS. 11-22.

In some embodiments, the transformation function implemented by the module 10 is bijective such that the amino acid sequence can be reconstructed from the numerical representation with knowledge of the transformation function. In some embodiments, other non-bijective transformation functions may be used to transform the amino acid sequence into the numerical representation. The other, non-bijective transformation functions may allow a better representation for the following modules, for example a neural network codification or compressive system that use protein sequence and other features.

In some embodiments, the interest inputs 2 provide the target or interest protein(s) as fragments or as a sequence of fragments. For example, when a particular fragment of the interest protein is desired, then the interest inputs 2 may include only the particular fragment(s) desired. In some embodiments, the interest inputs 2 provide the fragment(s) or sequence(s) of the target protein as a numerical representation. When the target protein information from the interest inputs 2 is provided as the numerical representation, then the module 10 may convert the other inputs 4 (e.g., the candidate proteins) to the numerical representation as described above.

The module 10 generates the protein fragments 15 comprising multiple numerical sequences that represent the protein fragments 15 of the original input candidate protein sequences from the other inputs 4. In some embodiments, the module 10 keeps the sequential order of the sequence of the protein fragments 15 the same in the generated output as compared to the original sequence in the other inputs 4.

In some embodiments, the system comprises a module 20. The module 20 may comprise one or more other modules, including a module 22, an internal representation 23, a concatenate block 25, and a module 24. In some embodiments, the internal representation 23 and the concatenate block 25 are optional. For example a language model can be pre-trained to learn a new presentation protein fragment and so forth for the module 24 and/or 30. In some embodiments, the model can be trained without the language model, which may degrade performance but still provide useful information.

The output protein fragments 15 of the module 10 are conveyed to the module 20. The module 20 may comprise a machine learning model (or similar) trained to predict a probability that every element within the input protein sequence of the candidate proteins received via the other inputs 4 appears in its current position given a context. Accordingly, the module 20 may assign different probability values to same or similar elements in the sequence where relative contexts are different between the same or similar elements. Thus, the machine learning models described herein may be trained to analyze the individual protein fragments 15 or sequences alone or in combination with any contextual information. Thus, in sum, the module 10 may generate the fragment(s) 15 of the candidate substitute protein that includes the feature(s) of interest or may return the entire candidate substitute protein that includes the feature(s) of interest in the target protein.

The module 20 may generate an interpretation of the fragments 15 and/or the entire candidate substitute protein and/or the target protein generated by the module 10. The module 20 may receive as an input the numerical sequences representing the protein fragments 15 (for example, the plurality of numerically codified sequences of the original candidate protein sequences from the other inputs 4) and the interest or target proteins and/or fragments from the interest inputs 2. These inputs of the module 20 may be processed by a module 22. The module 22 generates an internal representation of the fragments 15 generated by the module 10 and/or the target protein or fragments from the interest inputs 2. The module 22 may encode every element of the input sequences (e.g., the protein fragments 15) contextually. For example, the module 22 encodes the elements of the input protein fragment 15 sequences based on relational information and/or evolutive information (for example, amino acid co-evolution, MSAs, and so forth) obtained during a training process for the module 20 (for example, to train the model or similar network). In some embodiments, the internal representation 23 may preserve information on the coevolution of amino acids MSA/PSSM based on the output of the module 22. This is given by the specific task of predicting the probability of an amino acid, in a position, given its context. The internal representation 23 is the internal representation generated by the module 22. The concatenate 25 represents a concatenation of the internal representation. In some embodiments, the machine learning model implemented or applied in or by the module 20 comprises a recurrent neural network. In some embodiments, the module 20 comprises a convolutional neural network with direct connections or “skip” connections within layers. In some embodiments, the module 20 comprises a mixture of various neural networks, artificial intelligence models, and so forth. In some embodiments, the module 20 is configured to learn contextual correlations of the elements or protein fragments 15 within the sequence using one or more of the networks and models described herein. The module 22 outputs a codification of the numerical sequences representing proteins and/or protein fragments (either or both interest protein and/or candidate proteins), shown as the internal representation 23. The internal representation 23 output by the module 22 feeds into a pre-trained neural network or similar model of module 24, described further below. The neural network or model of the internal representation may learn certain frequencies of amino acids and other features of the amino acids and corresponding proteins. These learned features may comprise or be used as inputs for following models as “evolutive” contextual information.

This new codification output by the module 22 and/or the internal representation is concatenated at the block concatenate 25 with the original amino acid numerical representation to be used as input by module 24.

In some embodiments, the module 24 receives the same inputs (e.g., the protein fragments 15) as the module 22 (for example, the numerical sequences representing the protein fragments 15 or a plurality of numerically codified sequences of the original input candidate protein sequences of the other inputs 4) and interest proteins and/or fragments from the interest inputs 2. In some embodiments, the inputs received by the module 24 include one or more different codifications as generated by one or more of the internal representations block 23 or the concatenate block 25. The application of one or both of the internal representation 23 block and the concatenate block 25 is optional and may be skipped between the processes of module 22 and 24. In some embodiments, the module 24 controls or performs compressions of the information (for example, inputs received by the module 24) into a multidimensional space that contains evolutive, sequential, structural, and functional information of the interest and candidate proteins and/or fragments. The module 24 may also comprise or applies a machine learning model trained with codified and/or concatenated information (for example, as concatenated by the concatenate block 25) of every module that precedes it (for example, module 10 and/or module 22). The module 24 may generate an output that contains all the encoded information in a new representation of size L.

For example, the module 24 receives inputs of evolutive information from the output of the module 22, which was previously trained in a separate manner. Though not shown, the module 24 may also receive encoded information of proteins and/or fragments (interest proteins from the interest inputs 2 and/or candidate proteins and/or fragments from the protein fragments 15 generated by the module 10), which may include functional and/or structural data. Based on these inputs, the module 24 may generate an output containing all the above mentioned information that is encoded in a latent space (for example, the encoded representation) of size L. For example, the output of the module 24 may comprise a representation of the sequential, evolutionary, and structural information of the interest or candidate protein. All of the information of the corresponding protein (include one or more of the sequential, evolutionary, and structural information) can be encoded in a single vector of the length of the protein sequence and a fixed number of dimensions (for example, 100).

In some embodiments, the module 24 comprises one or more of a recurrent neural network with multiple layers stacked together in a bidirectional manner and a convolutional neural network with multiple layers stacked with “skip” connections within layers that was trained to predict the function, secondary structure, tertiary structure, and/or contact maps of proteins (for example, the interest and/or candidate proteins). As such, the module 24 may predict protein features because the information used by the model or networks of the module 24 to learn intermediate representation is useful in inferring such protein features. For example, because the fragments 15 generated by the module 10 relate to the structural similarities between the target or interest protein and the candidate protein, function of the candidate protein or the candidate protein fragment can be extrapolated, for example by the module 24. The features prediction is used to optimize a protein based on the collected fragments. When the features of proteins can be compared, different variants of proteins (e.g., in the plurality of candidate proteins) can be compared to select the one(s) that out-stand in the feature of interest (e.g., that share the most features of interest with the target protein). Furthermore, the module 24 can infer features because it was trained to predict secondary tasks. Accordingly, the model can “retain” that information in the latent space.

In some embodiments, the module 24 generates two sets of encoded representations, the encoding interest representations 27 based on the interest proteins and/or fragments of the interest inputs 2 and the encoding candidate (or other) representations 29 based on the candidate proteins and/or fragments 15 (which are potential proteins that may be used as ingredients with the same function as the interest proteins). The module 24 predicts the protein contact maps and secondary structures as two different tasks, the protein design model uses another two Bi-LSTM stacked layers, each with 1,024 hidden dimensions and a projection, on to the initial Language Model or module 22. For the contact maps and secondary structure predictions, the protein design model used cross-entropy as a loss function and trained the models for five epochs using the SCOP database filtered at 40% identity, which included more than 30,000 structures. Therefore module 24 allows the system to capture structural information from the inputs (module 22 only considers amino acid sequence information).

In some embodiments, the module 20 receives protein amino acid sequence information and outputs a numerical representation (embedding tensor) containing protein structural information inferred from the amino acid sequence. More specifically, the module 20 changes the format of the fragments or target protein inputs into the encoding interest representations 27 and the encoding others representation 29. To generate the outputs of the module 20 mentioned above, module 22 encodes the protein sequence received from the module 10 or directly from the inputs 2 and 4 to a numerical representation (for example, internal representation 23). This internal representation 23 can be concatenated to collect evolutive information (such as a multiple protein sequence alignment), creating the concatenate 25. This information is passed to module 24, which is the module that outputs structural information (such as protein contact maps, and protein secondary structure) to the module 30. RAs described below, both these sets of encoded representations are inputs to the module 30.

In some embodiments, the system 1 further comprises a module 30. The module 30 may receive inputs of the “encoding interest” representations 27 and “encoding others” representation 29. In some embodiments, the module 30 controls the comparison of or compares interest proteins or fragments with the candidate proteins or fragments. In some embodiments, the module 30 comprises one or more of a recurrent neural network, a convolutional neural network with “skip” connections, or a fully connected neural network that learns to generate a hierarchical score representing the similarity within the compared proteins based on sequential, structure, folding and functional similarities between the compared proteins. The machine learning model applied by the module 30 may generate the similarity rank 32 for the pair of target protein and candidate protein being analyzed or processed. In some embodiments, the similarity rank 32 is stored for later evaluation or integrated into a report or presented via a user interface. The report may include details of the target protein and feature of interest, the candidate protein, any numerical or coding representations thereof, and so forth. Thus, the module 30 compares the encoding interest representations 27 and the encoding others representations 29 (e.g., compares the target protein and the candidate protein with respect to the desired feature in the target protein) to generate a score indicative of how similar the candidate protein and the target protein are, relative to the feature of interest.

The system of FIG. 1 may be used to look for functional proteins (for example, animal-free, plant-based, and/or synthetic proteins) that are analogous to proteins of animal origin. For example, the protein of interest may be gelatin (a protein of animal origin) and candidate substituted proteins may be synthetic or plant-based proteins that are functionally similar to the animal-based gelatin.

FIG. 2 is a block diagram of a module 10 of the system of FIG. 1 that is configured to split or fragment proteins. In some embodiments, the module 10 may receive an input of candidate substitute proteins. As described above, the module 10 may fragment the input candidate substitute proteins into functional domains and/or fragments that correspond to the interest protein's size. The module 10 may generate as its output the fragments of the input candidate substitute proteins.

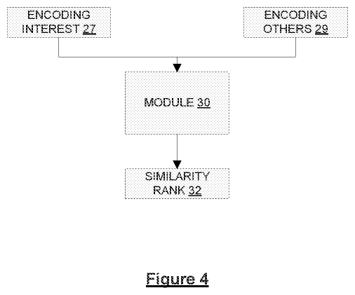

FIG. 3 is a block diagram of a module 20 of the system of FIG. 1 that is configured to encode protein information in a numerical codification. In some embodiments, the module 20 receives the fragments of the input candidate substitute proteins as an input. The module 20 may also receive the interest protein (or fragments thereof) as an input. The module 20 may then encode the fragments of the input candidate substitute proteins and the interest protein in a multidimensional space that contains the sequential, structural and functional information of the inputs. The module 20 may output the encoded proteins and/or fragments.

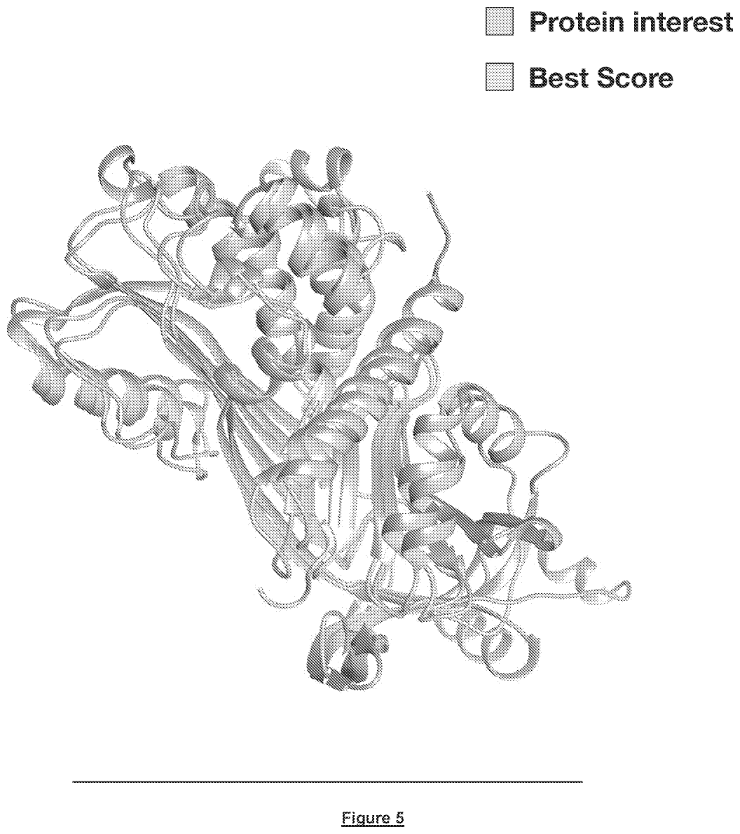

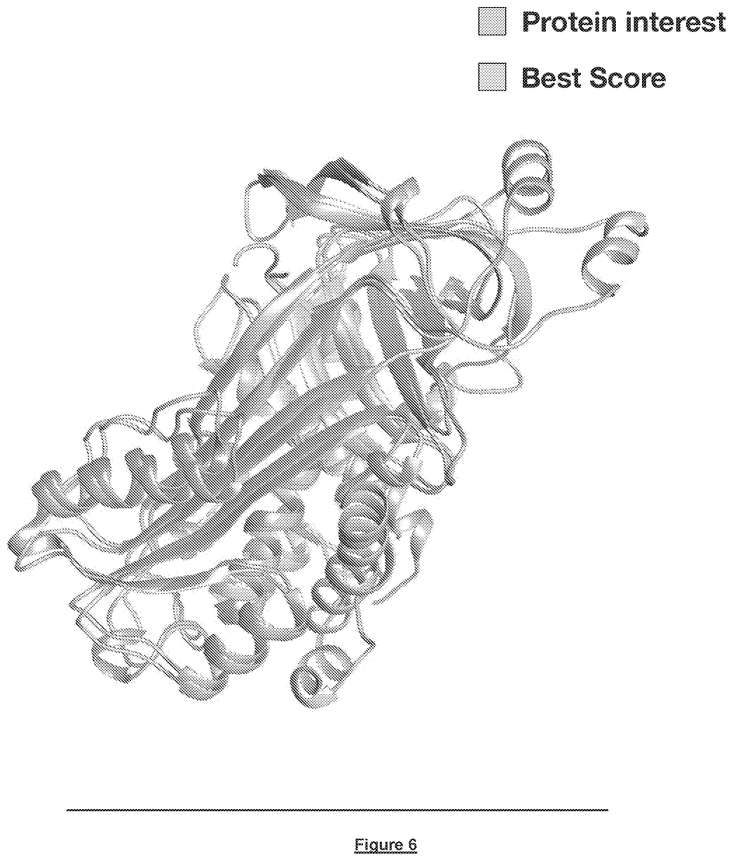

FIG. 4 is a block diagram of a module 30 of the system of FIG. 1 that is configured to compare and hierarchically rank protein information using a numerical coding and generate a similarity rank 32. In some embodiments, the module 30 receives the encoded proteins and/or fragments that are output by the module 20. The module 30 may compare each protein and/or fragment (for example, compare the protein or protein fragment of the interest protein with the protein or fragment of the candidate protein) with a similarity metric in the multidimensional space. In some embodiments, the module 30 may also hierarchize the compared proteins and/or fragments from the most similar to the least similar (or according to any other metrics). In some embodiments, hierarchizing comprises assigning a score or similar value to the compared proteins and/or fragments. The module 30 may select those proteins with the best score (for example, providing a defined, fixed number of candidates or those proteins whose score exceeds a threshold). The candidate proteins and/or fragments of the selected proteins may constitute the highest matched candidate proteins and/or fragments that best correlate with the interest protein or fragments. These selected proteins and/or fragments may then be bioinformatically simulated and/or synthesized in the laboratory to verify the activity and fulfillment of the desired function of the interest protein. In some embodiments, the similarity rank 32 is presented for viewing by a user along with one or more of the target protein (or fragment thereof) and the candidate protein (or fragment thereof) structures for visual comparison or viewing (as shown in FIGS. 5 and 6). In some embodiments, the module 30 includes a ranker that can order the candidate proteins from a best score to a worst score. For example, using the similarity scores between the sequences and fragments of sequences, the modules 30 can order the plurality of sequences from the most similar to the least similar, and take the top K to simulate or synthesize in lab.

FIG. 5 is a first view of a pairing of protein structures including an animal-based target protein (e.g., from the interest inputs 2) and a corresponding plant-based protein as generated or created by the system 1 of FIG. 1 based on a crystal and computation prediction. The result shows that the top-score protein is structurally similar to the target protein meaning they have similar functional proteins but coming from different sources.

FIG. 6 is a second view of the pairing of protein structures of FIG. 5. The result shows that the top-score protein is structurally similar to the target protein meaning they have similar functional proteins but coming from different sources. Differences can be observed at the sequence level, where both proteins present an identity percentage lower than 30%. These proteins can be considered as analogous proteins.

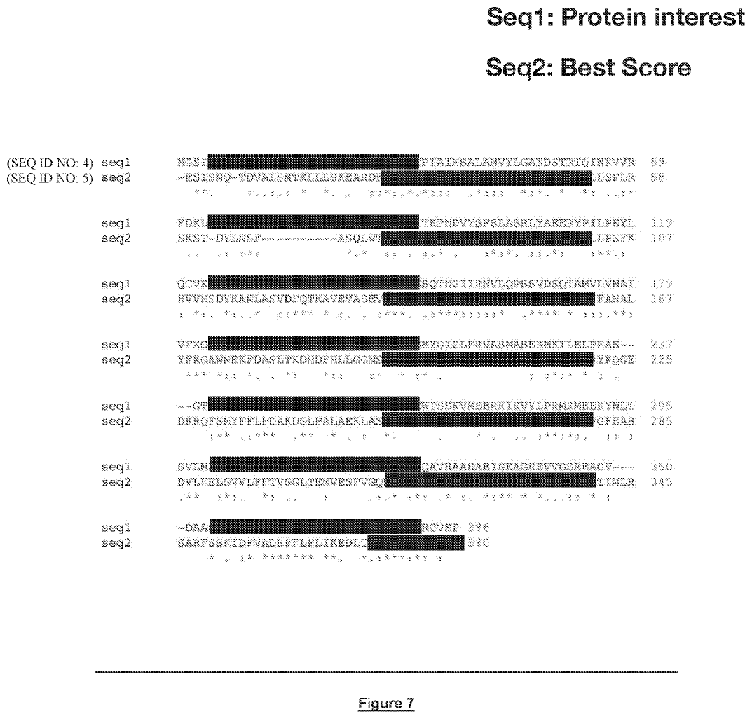

FIG. 7 shows an alignment of the two protein sequences of FIGS. 5 and 6 that includes a protein of animal origin and a corresponding, animal-free protein generated by the system of FIG. 1. Results shows that both sequences are not homologous but some key residues are conserved.

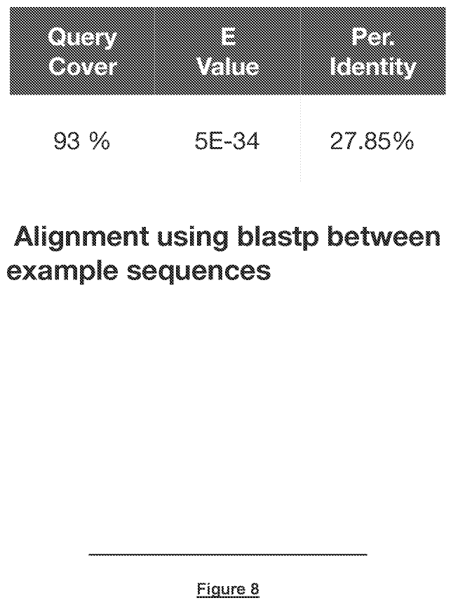

FIG. 8 shows a table of results of a comparison of the animal-based protein with the animal-free protein of FIGS. 5, 6, and 7. This result using BLASTp shows that the pair of proteins are not homologous, and could be considered as analogous proteins. Query coverage may correspond to length of an alignment with respect to a query sequence. E-value may correspond to an expectation value and be related to a number of hits one can expect to see by chance when searching a database. Percentage (per.) identity may correspond to a percentage of sequence identity that exists between both protein sequences (e.g., the target protein sequence and the candidate protein sequence).

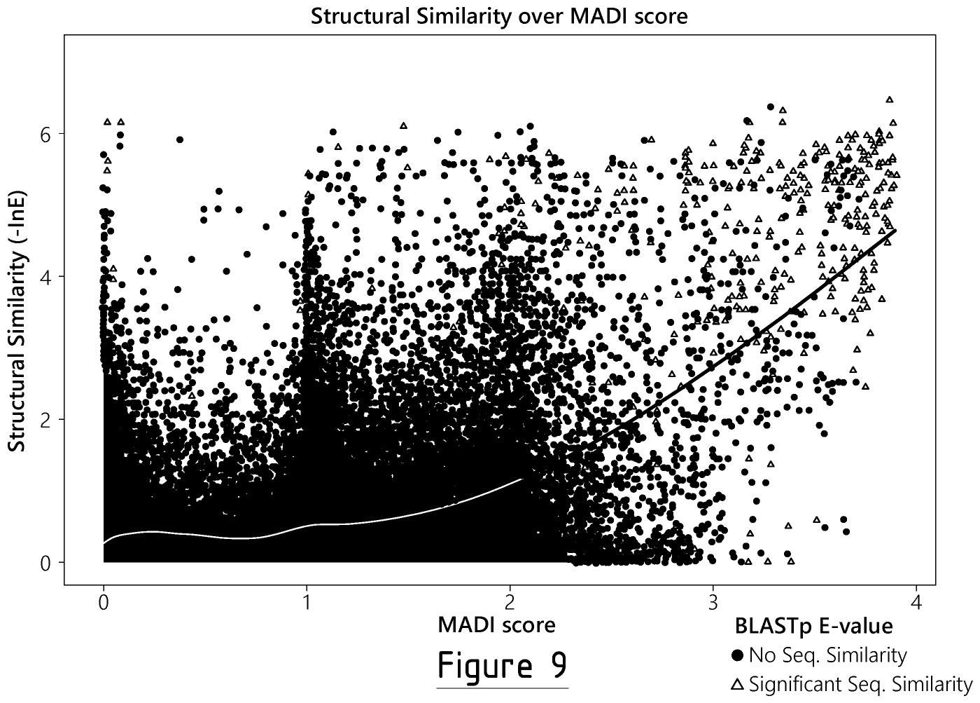

FIG. 9 shows a plot of comparisons between the animal-based and corresponding animal-free protein structures as stored in a protein data bank. Results show that the system predicted similarity (similarity score) allows selection of proteins with similar functional properties but with different amino acid sequences that cannot be identified with other methods. Additional Example Implementations of the System

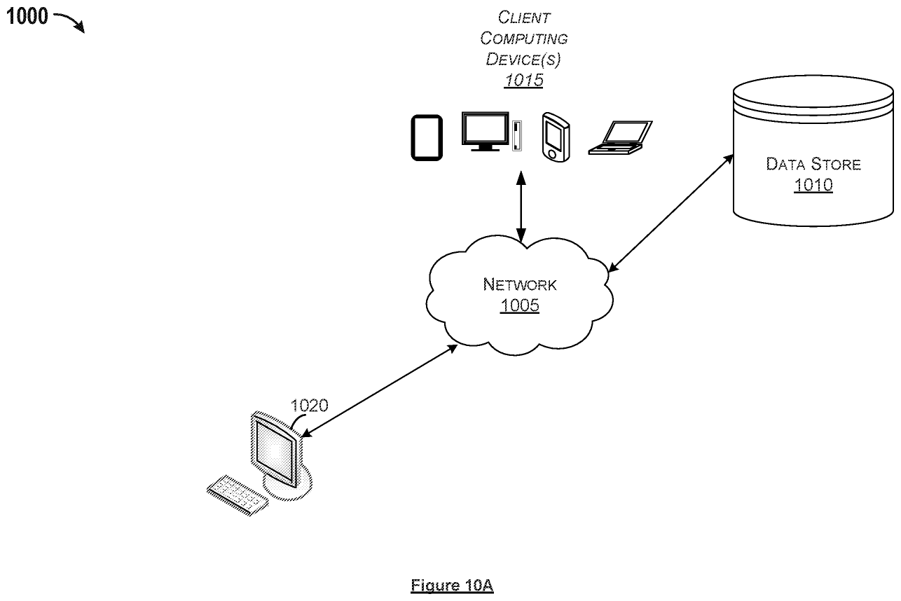

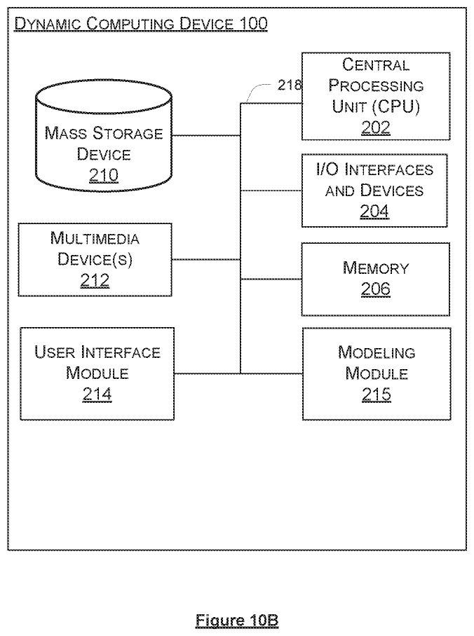

FIGS. 10A and 10B illustrate embodiments of a dynamic modelling system used to identify, score, and generate proteins that comprise a desired feature of a target protein.

### System Overview

FIG. 10A illustrates one embodiment of a networked system 1000 that allows users to dynamically identify and generate proteins having corresponding features as a target protein according to system 1 of FIG. 1.

The system 1000 of FIG. 10A comprises a dynamic computing device 1020 interfacing with a network 1005, a data store 1010, and one or more client computing devices 1015. Additionally, communication links are shown enabling communication among the components of system 1000 via the network 1005. The dynamic computing device 1020 may be communicatively coupled to another device used to generate proteins based on results determined or identified by the dynamic computing device 1020. Furthermore, the data store 1010 described herein may be separated into multiple data stores integrated with the system 1000. In some embodiments, two or more of the components described above may be integrated. The system 1000 (or one of the components shown therein) may be used to implement systems and methods described herein.

In some embodiments, the network 1005 may comprise any wired or wireless communication network by which data and/or information may be communicated between multiple electronic and/or computing devices. The wireless or wired communication network may be used to interconnect nearby devices or systems together, employing widely used networking protocols. The various aspects described herein may apply to any communication standard, such as a wireless 802.11 protocol. The dynamic computing device 1020 may comprise any computing device configured to transmit and receive data and information via the network 1005. In some embodiments, the computing device 1020 may include or have access to one or more databases (for example, the data store 1010) that include various target and/or candidate proteins or fragments (for example, of the interest inputs 2 and/or the other inputs 4). In some embodiments, the dynamic computing device 1020 may be accessible locally as well as remotely via the network 1005 (for example, via one of the client computing device 1015). The dynamic computing device 1020 may create the customized outputs described herein to identify candidate proteins (or fragments thereof, for example of the other inputs 4) that share features with a target protein (for example, one of the interest inputs 2) based on similarity of the candidate protein (or fragment thereof of the protein fragments 15) and the target protein.

The data store 1010 may comprise one or more databases or data stores and may also store data regarding any of the target proteins, candidate proteins, fragments of the target proteins or candidate proteins, similarity scores, representations of proteins, and so forth. Using the example use case, the data store 1010 may comprise the similarity scores generated by the system 1 or the numerical representations generated by the system 1.

The computing devices 1020 or 1015 may comprise any computing device configured to transmit and receive data and information via the network 1005. In some embodiments, the computing device 1015 may be configured to control the dynamic computing device 1020 (or vice versa) to control the system 1 of FIG. 1. In some embodiments, the one or more computing devices 1020, 1015 may comprise mobile or stationary computing devices. In some embodiments, the computing device 1020, 1015 may be integrated into a single terminal or device. In some embodiments, the computing devices 1015 may be used by users to access the network 1005 and access the computing device 1020 remotely.

The dynamic computing device 1020 may process proteins as described above in relation to system 1 of FIG. 1. In some embodiments, the dynamic computing device 1020 may dynamically generate, train, and/or apply one or more machine learning models as described herein to perform one or more of the functions described herein. For example, the dynamic computing device 1020 may generate, train, and apply the machine learning models described above to identify protein sequences or fragments thereof of a set of candidate proteins (for example, the other inputs 4) that have features that correspond to desired features in a target protein sequence or fragment (for example, the interest input 2). In some embodiments, the development of the model may include developing a set of heuristic rules, filters, and/or electronic screens to determine, /or identify, and/or predict which proteins from the candidate proteins would provide similar desired features as the target protein. In some embodiments, the dynamic computing device 1020 may automatically adjust any machine learning model to meet pre-selected levels of accuracy and/or efficiency.

In some embodiments, the different models applied by the system 1 are trained independently. For example, the models applied by the module 10 to generate the fragments may be trained to be able to fragment input proteins that have more than one functional domain into protein fragments that have only one functional domain. The model applied by the module 10 may be trained by or comprise a supervised model(s) provided with a protein sequence and apply the model to determine which are the sites in the protein where a functional domain begins or ends, as the cutting sites are only punctual. As described above, these sites may have a tolerance range of +/−20 amino acids.

In some embodiments, the models applied by the module 22 may comprise a language model. The language model may be trained using and/or comprise a semi-supervised model(s) in which a window of the protein is provided and the model is applied to predict the next amino acid. In some embodiments, the model is provided masked amino acids which the model predicts given its context.

In some embodiments, the module 24 applies a structural task model, where given the sequence of a protein, the model is applied to predict structural information of the protein. The predicted structural information can be secondary structure, contact maps, and/or three-dimensional structure.

In the module 30, the similarity score is identified by apply a corresponding model. The model is provided with two pairs of values (for example, corresponding to the two sequences, interest protein and candidate protein)) and the model predicts how similar the two sequences are to each other. The pairs may be categorized according to the similarity score, for example as having a same family, same superfamily, same folding, and so forth. Each of these models can be retrained with experimental results, in the same way as they were previously trained but adding the new data to update the weights of the model.

In some embodiments, the dynamic computing device 1020 may be adaptive to data from the data store 1010 or from users that is constantly changing. For example, the inputs received from the user (for example, via the user interface module 214 or the I/O interfaces and devices 204 in FIG. 10B) may be different for each user. Using an example use case, one user may be interested in a first target protein feature or a first set of candidate proteins, whereas another user may be interested in a different target protein feature or a second set of candidate proteins, and so forth. Accordingly, the data obtained from the data store 1010 and the user via the user interface 214, etc., will likely be constantly changing. Thus, the processing and/or model generation will change for each user, target protein, feature of interest, candidate protein, and so forth. Accordingly, the dynamic computing device 1020 may dynamically generate models to handle constantly changing data and requests.

In various embodiments, large amounts of data are automatically and dynamically calculated interactively in response to user inputs or requests, and the calculated data is efficiently and compactly presented to a user or used by other components in the system 1000. Thus, in some embodiments, the data processing and generating of models and outputs described herein are more efficient as compared to previous data processing and model generation in which data and models are not dynamically updated and compactly and efficiently presented to the user in response to interactive inputs.

The various embodiments of interactive and dynamic data processing and output generation of the present disclosure are the result of significant research, development, improvement, iteration, and testing. This non-trivial development has resulted in the modeling and output generation described herein, which may provide significant efficiencies and advantages over previous systems. The interactive and dynamic modeling, user interfaces, and output generation include improved human-computer and computer-computer interactions that may provide reduced workloads, improved predictive analysis (for example, of candidate proteins that share features of interest with target proteins), and/or the like. For example, output generation via the interactive user interfaces described herein may provide an optimized display of time-varying protein-related similarity rank, representation, and other information and may enable a user to more quickly access, navigate, assess, and digest such information than previous systems.

In some embodiments, output data or reports may be presented in graphical representations, such as visual representations, such as charts, spreadsheets, and graphs, where appropriate, to allow the user to comfortably review the large amount of data and to take advantage of humans' particularly strong pattern recognition abilities related to visual stimuli. For example, the sequence structures shown in relation to FIGS. 5 and 6 or the sequence shown in FIG. 7 may be presented to as part of the visual representation to allow for user review. In some embodiments, the system may present aggregate quantities, such as totals, counts, and averages, or score information (for example, rankings and so forth). The system may also utilize the information to interpolate or extrapolate, for example, forecast, future developments.

Further, the models, data processing, and interactive and dynamic user interfaces described herein are enabled by innovations in efficient data processing, modeling, interactions between the user interfaces, and underlying systems and components. For example, disclosed herein are improved methods of receiving user inputs and protein inputs, translation and delivery of those inputs to various system components, automatic and dynamic execution of complex processes in response to the input delivery, automatic data acquisition, automatic interaction among various components and processes of the system, and automatic and dynamic report generation and updating of the user interfaces. The interactions and presentation of data via the interactive user interfaces described herein may accordingly provide cognitive and ergonomic efficiencies and advantages over previous systems.

Various embodiments of the present disclosure provide improvements to various technologies and technological fields. For example, as described above, existing data storage and processing technology (including, for example, in memory databases) is limited in various ways (for example, manual data review is slow, costly, and less detailed; data is too voluminous; and so forth), and various embodiments of the disclosure provide significant improvements over such technology. Additionally, various embodiments of the present disclosure are inextricably tied to computer technology. In particular, various embodiments rely on detection of user inputs via user interfaces, acquisition of data based on those inputs, identifying representations of protein data, modeling of data to generate dynamic outputs based on those user and protein inputs, automatic processing of related electronic data, and presentation of output information via interactive user interfaces or reports. Such features and others (for example, processing and analysis of large amounts of electronic data) are intimately tied to, and enabled by, computer technology, and would not exist except for computer technology. For example, the interactions with data sources and displayed data described below in reference to various embodiments cannot reasonably be performed by humans alone, without the computer technology upon which they are implemented. Further, the implementation of the various embodiments of the present disclosure via computer technology enables many of the advantages described herein, including more efficient interaction with, and presentation of, various types of electronic data.

### Dynamic Modeling System

FIG. 10B is a block diagram corresponding to an aspect of a hardware and/or software component of an example embodiment of a dynamic computing device 100 of the system 1000 of FIG. 10A. The hardware and/or software components, as discussed below with reference to the device 100 may be included in any of the devices 1015 or the device 1020 of the system 1000 (for example, the dynamic computing device 1020 or the computing devices 1015, and so forth). These various depicted components may be used to implement the systems and methods described herein.

In some embodiments, certain modules described below, such as the modeling module 215 or a user interface module 214 included with the device 100 may be included with, performed by, or distributed among different and/or multiple devices of the system 1000. For example, certain user interface functionality described herein may be performed by the user interface module 214 of various devices such as the computing device 102 and/or the one or more computing devices 106.

In some embodiments, the various modules described herein may be implemented by either hardware or software. In an embodiment, various software modules included in the device 100 may be stored on a component of the device 100 itself (for example, a local memory 206 or a mass storage device 210), or on computer readable storage media or other component separate from the device 100 and in communication with the device 100 via the network 1005 or other appropriate means.

The device 100 may comprise, for example, a computer that is IBM, Macintosh, or Linux/Unix compatible or a server or workstation or a mobile computing device operating on any corresponding operating system. In some embodiments, the device 100 interfaces with a smart phone, a personal digital assistant, a kiosk, a tablet, a smart watch, a car console, or a media player. In some embodiments, the device 100 may comprise more than one of these devices. In some embodiments, the device 100 includes one or more central processing units (“CPUs” or processors) 202, I/O interfaces and devices 204, memory 206, the dynamic modeling module 215, a mass storage device 210, a multimedia device 212, the user interface module 214, and a bus 218.

The CPU 202 may control operation of the device 100. The CPU 202 may also be referred to as a processor. The processor 202 may comprise or be a component of a processing system implemented with one or more processors. The one or more processors may be implemented with any combination of general-purpose microprocessors, microcontrollers, digital signal processors (“DSPs”), field programmable gate array (“FPGAs”), programmable logic devices (“PLDs”), controllers, state machines, gated logic, discrete hardware components, dedicated hardware finite state machines, or any other suitable entities that can perform calculations or other manipulations of information.

The I/O interface 204 may comprise a keypad, a microphone, a touchpad, a speaker, and/or a display, or any other commonly available input/output (“I/O”) devices and interfaces. The I/O interface 204 may include any element or component that conveys information to the user of the device 100 (for example, a user requesting the candidate protein with the corresponding feature of interest) and/or receives input from the user. In one embodiment, the I/O interface 204 includes one or more display devices, such as a monitor, that allows the visual presentation of data to the user. More particularly, the display device provides for the presentation of GUIs, application software data, websites, web apps, and multimedia presentations, for example, the protein representations of FIGS. 5-7.

In some embodiments, the I/O interface 204 may provide a communication interface to various external devices. For example, the device 100 is electronically coupled to the network 1005 (FIG. 10A), which comprises one or more of a LAN, WAN, and/or the Internet. Accordingly, the I/O interface 204 includes an interface allowing for communication with the network 1005, for example, via a wired communication port, a wireless communication port, or combination thereof. The network 1005 may allow various computing devices and/or other electronic devices to communicate with each other via wired or wireless communication links.

The memory 206, which includes one or both of read-only memory (ROM) and random access memory (“RAM”), may provide instructions and data to the processor 202. For example, data received via inputs received by one or more components of the device 100 may be stored in the memory 206. A portion of the memory 206 may also include non-volatile random access memory (“NVRAM”). The processor 202 typically performs logical and arithmetic operations based on program instructions stored within the memory 206. The instructions in the memory 206 may be executable to implement the methods described herein (e.g., generate, train, and/or apply the machine learning models described herein). In some embodiments, the memory 206 may be configured as a database and may store information that is received via the user interface module 214 or the I/O interfaces and devices 204.

The device 100 may also include the mass storage device 210 for storing software or information (for example, the generated models or data obtained to which the models are applied, and so forth. Software shall be construed broadly to mean any type of instructions, whether referred to as software, firmware, middleware, microcode, hardware description language, or otherwise. Instructions may include code (for example, in source code format, binary code format, executable code format, or any other suitable format of code). The instructions, when executed by the one or more processors, cause the processing system to perform the various functions described herein. Accordingly, the device 100 may include, for example, hardware, firmware, and software, or any combination therein. The mass storage device 210 may comprise a hard drive, diskette, solid state drive, or optical media storage device. In some embodiments, the mass storage device 210 may be structured such that the data stored therein is easily manipulated and parsed.

As shown in FIG. 10B, the device 100 includes the modeling module 215. As described herein, the modeling module 215 may dynamically generate one or more machine learning models described herein for processing data obtained from the data stores or the user or the proteins. In some embodiments, the modeling module 215 may also apply the generated models to the data. In some embodiments, the one or more models may be stored in the mass storage device 210 or the memory 206. In some embodiments, the modeling module 215 may be stored in the mass storage device 210 or the memory 206 as executable software code that is executed by the processor 202. This, and other modules in the device 100, may include components, such as hardware and/or software components, object-oriented software components, class components and task components, processes, functions, attributes, procedures, subroutines, segments of program code, drivers, firmware, microcode, circuitry, data, databases, data structures, tables, arrays, and variables. In the embodiment shown in FIG. 10B, the device 100 is configured to execute the modeling module 215 to perform the various methods and/or processes as described herein.

In some embodiments, though not shown, a report module may be configured to generate a report, notification, or output mentioned and further described herein. In some embodiments, the report module may utilize information received from the device 100, the data acquired from the data stores, and/or the dynamic computing device 1020 or the user of the computing device 106 or the proteins of the datastore 1010 to generate the report, notification, or output. For example, the device 100 may receive information for identifying a protein from a set of candidate proteins that best share or replicate a feature of a target protein. The device 100 may utilize the systems and methods described herein, for example the system 1 and modules, etc., described in relation thereto, to generate results including the similarity rank and/or visual representations of the target and candidate proteins of FIGS. 5-7. In some embodiments, the generated report, notification, or output may comprise the similarity rank and/or visual representations. In some embodiments, the report module may include information received from the user in the generated report, notification, or output (for example, the feature of interest, etc.).

The device 100 also includes the user interface module 214. In some embodiments, the user interface module 214 may also be stored in the mass storage device 210 as executable software code that is executed by the processor 202. In the embodiment shown in FIG. 10B, the device 100 may be configured to execute the user interface module 214 to perform the various methods and/or processes as described herein.

The user interface module 214 may be configured to generate and/or operate user interfaces of various types. In some embodiments, the user interface module 214 constructs pages, applications or displays to be displayed in a web browser or computer/mobile application. In some embodiments, the user interface module 214 may provide an application or similar module for download and operation on the computing device 1020 and/or the computing devices 1015, through which the user may interface with the device 100 to obtain the desired report or output. The pages or displays may, in some embodiments, be specific to a type of device, such as a mobile device or a desktop web browser, to maximize usability for the particular device. In some embodiments, the user interface module 214 may also interact with a client-side application, such as a mobile phone application, a standalone desktop application, or user communication accounts (for example, e-mail, SMS messaging, and so forth) and provide data as necessary to display vehicle equity and prequalification determinations.

The various components of the device 100 may be coupled together by the bus system 218. The bus system 218 may include a data bus, for example, as well as a power bus, a control signal bus, and a status signal bus in addition to the data bus. In different embodiments, the bus could be implemented in Peripheral Component Interconnect (“PCI”), Microchannel, Small Computer System Interface (“SCSI”), Industrial Standard Architecture (“ISA”) and Extended ISA (“EISA”) architectures, for example. In addition, the functionality provided for in the components and modules of the device 100 may be combined into fewer components and modules or further separated into additional components and modules than that shown in FIG. 10B.

As described herein, the modules 10, 20, 22, 24, and 30 may comprise a processor, a microprocessor, or similar computing structure or circuit. In some embodiments, the modules 10, 20, 22, 24, and 30 may be configured for execution by a processor to perform, in whole or in part, any or all of the process or systems discussed above, such as those described above with respect to FIGS. 1-4. In some embodiments, the methods and systems described herein (e.g., the system 1) may be coupled to a device for generating a high-dimensional numerical representation of a protein sequence from a physical protein structure. This high-dimensional representation contains crucial information used, as described herein, for identifying similarities and differences between proteins.

### Additional Embodiments

Protein sequences are highly dimensional, which presents problems for the optimization and study of sequence-structure relations in the proteins. While the intrinsic degeneration of protein sequences can be difficult to follow, the continued discovery of new protein structures has shown that there is a convergence in terms of possible folds that proteins can adopt, such that proteins with sequence identities lower than 30% may still fold into similar structures as those with sequence identities higher than 30%. Because proteins share a set of conserved structural motifs, machine learning algorithms can play an essential role in the study of sequence-structure relationships. Deep-learning neural networks may be an important tool in the development of new techniques of predicting and generating proteins, such as protein modeling and design, and these techniques continue to gain power as new algorithms are developed and as increasing amounts of data are released every day. A newly developed and trained deep-learning model designs analog protein structures using representations, where the deep-learning model learns based on evolutionary and structural information contained in more than 20 million protein sequences. Capabilities of this model are tested by creating de novo variants of an antifungal peptide, having sequence identities of 50% or lower relative to the wild-type (WT) peptide. In silico approximations, such as molecular dynamics, new variants and the WT peptide can successfully bind to a chitin surface with comparable relative binding energies. These results are supported by in vitro essays, where the de novo designed peptides showed antifungal activity that equalled or exceeded the WT peptide.

Proteins are one of the most interesting, widespread, and highly studied macromolecules due to their highly functional features. Proteins differ in their primary structure, which consists of a sequence of amino acids; this sequence strictly determines the spontaneous folding patterns and spatial arrangements that characterize their three-dimensional structures, which then define their diverse molecular functions. Due to their diverse functional properties, proteins are of interest for a wide range of industrial applications. Rising interest from industry, combined with the increasing number of protein databases and structures and the development of more efficient computational algorithms, has led to the design of different pipelines for predicting the tertiary or quaternary structure and function of a protein from its primary structure.

The vast diversity of protein structures is made possible by the effectively infinite number of possible combinations of twenty natural amino acids. This structural diversity has enabled the evolution of multiple functions responsible for most biological activities. Protein structures are usually determined by techniques such as NMR, X-ray crystallography, and cryo-electron microscopy, but in silico structure prediction methods have been shown to be an important alternative when experimental limitations exist. Protein structure prediction pipelines can follow a template-based or template-free approach (or a combination of both). In the template-based approach, protein structure decoys are built based on previously known protein structures, whereas in the template-free approach, no structural templates are needed and new protein folds can be explored if desired. One of the most widely-used approaches is fragment-based protein structure prediction. In this approach, decoys are built based on libraries of protein fragments with known structures (e.g. Rosetta), with a search guided on angle torsions and secondary structures.

Two general conventions exist for grouping proteins based on their structural similarity: i) homologous proteins inherit similarities from common ancestors, maintaining similar sequences and structures; and ii) analogous proteins have similar structures, given the limited local energy minima of their three-dimensional arrangements, but they do not necessarily maintain similar sequences with an evolutionary connection. There are many examples of homologous proteins (i.e., proteins that share a common ancestor) as well as many tools for finding their evolutionary relationships and predicting their structure. A classic example of protein homology is the TIM barrel fold, which is present in roughly 10% of all enzymes. Moreover, homologous protein structures are widely classified in different databases such as CATH, SCOP, and Pfam, while only one database exists for analogous motifs. In general, fewer studies attempt to recognize structural analogs. Some examples of analogous structures are the hybrid motif βαβββ from the oligopeptide-binding protein OPPA in Salmonella typhimurium (PDB: 1B05), which is analogous to the core motif βαβββ from the antibiotic resistance protein FosA in Pseudomonas aeruginosa (PDB: 1NKI), and the artificial nucleotide-binding protein (ANBP, PDB: 1UW1), which is analogous to the treble clef zinc-binding motif.

There are clear limitations to the available approaches for searching for structural analogs, and it has previously been suggested that more accurate statistical estimates are needed in order to identify similarities that are due to analogy rather than homology. It is therefore important to develop new alternatives that improve the conventional homology-based structure prediction approaches by also detecting analogous protein structures. One such alternative is a pipeline for using docking-based domain assembly simulations to assemble multi-domain protein structures, with interdomain orientations determined based on the distance profiles from analogous protein templates.

Recently, machine learning algorithms have received major recognition as an approach to predicting important sequence-structure relationships. Deep-learning (DL) strategies are neural networks with internal processing layers that can be trained to recognize patterns in large and complex data. DL strategies have been used for various protein applications, including the prediction of protein secondary structure and subcellular localization; the prediction of protein contact maps, homology and stability; protein design, such as the prediction of protein sequences based on protein structures and the design of metalloproteins; and the prediction of protein folding, among several other applications. It is therefore of great interest to develop new tools that can accurately predict new protein sequence-structure relationships. Such tools will open doors for the automated search, prediction, and design of analog or low-homology proteins. These tools could additionally be used for other applications, such as the design of new protein structures and functions and the labeling of the dark proteome, in order to enrich scientific knowledge in these areas.

Here, the system 1 described above can apply a new DL model that allows for the design of analog proteins with low sequence homology using a representations learning approach based on both evolutionary and structural information. This information was provided to the model implicitly through large databases of unlabeled protein sequences (such as Pfam) and of labeled structures (such as PDB and SCOPe). The DL model may use language models that have revolutionized natural language processing (NLP), where the baseline is a semi-supervised Bidirectional Long/Short-Term Memory (Bi-LSTM) that is trained to predict the most probable next word given its context. These language models can extract biochemical and evolutionary information but lack structural information. To solve this, the system applies another model that allows the initial language model to learn structural information by predicting protein contact maps and secondary structures. By stacking these models, the system obtains a vector representation (embedding) that allows us to find both structural and sequence similarities. Based on the similarity between these vector representations, the system can search for and design proteins with desired structures and activities. Some of these structures and activities have not been explored by nature, given that homologous proteins with more than 50% sequence identity are difficult to find. The DL model can capture both homology and analogy and could potentially improve template-based protein structure predictions, as well as other protein prediction tasks such as protein functionality.

The protein design model (for example, as represented by the system 1 and described with relation to FIGS. 1-10B above) can generate new de novo antifungal peptides based on target features in the target or interest sequence. Antifungal peptides are receiving increasing interest due to their potential applications in diverse industries such as food manufacturing, agriculture, cosmetics, and therapeutics, where these antifungal peptides offer an attractive and useful replacement to current chemical alternatives.

### Methods of Designing Functional De Novo Antimicrobial Proteins

Language Model (for example, as implemented or represented by module 22 in FIGS. 1-4 above). The protein design model of system 1 employs a Bi-LSTM encoder as a language model (e.g., module 22). The module 22 may takes a protein sequence where each amino acid is a token (i.e., an instance of a sequence of characters in some particular document that are grouped together as a useful semantic unit for processing) and represents the protein as a vector of the same length. The architecture of or represented by the module 22 may consist of two Bi-LSTM layers with 1,024 hidden units in each layer, with a linear projection into the 20 amino acid prediction. The model is trained for six epochs with the ADAM optimizer, using a learning rate of 1*10⁻³ and a training batch size of 32 in a GPU V100. The module 22 was trained based on more than 21 million protein sequences obtained from Pfam. The module 22 uses a classical next-token prediction task with cross-entropy as a loss function.

Structural Features Prediction (for example, as implemented or represented by the module 24 in FIGS. 1-4 above). To predict protein contact maps and secondary structures as two different tasks, the protein design model of system 1 uses another two Bi-LSTM stacked layers, each with 1,024 hidden dimensions and a projection, on to the initial module 22. For the contact maps and secondary structure predictions, the protein design model used cross-entropy as a loss function and trained the models for five epochs using the SCOP database filtered at 40% identity, which included more than 30,000 structures.

Molecular dynamics simulations. The protein suggested by the system 1 (for example, the candidate protein having the highest score of the plurality of candidate proteins compared using the system 1) can be further analyzed using classic computational biology tools, such as molecular dynamics simulations. Atomistic molecular dynamics (MD) simulations were run for the wild-type AC2 peptide (SEQ ID NO: 1—VGECVRGRCPSGMCCSQFGYCGKGPKYCGR, PDB: 1ZUV, with tryptophan 18 mutated back to phenylalanine), as well as for the two de novo design variants, DNv1-AC2 (SEQ ID NO: 2—VQDWCGNDCSAKECCKRDGYCGWGVDYCGG) and DNv2-AC2 (SEQ ID NO: 3—KRCGSQAGCPNGHCCSQYGFCGFGPEYCGR). Peptide models for DNv1-AC2 and DNv2-AC2 were based on the best matches from a homology search conducted using the HHpred bioinformatics toolkit. The proteins that were used as templates were PDB 2KUS and 1MMC, which correspond to the antifungal peptides Sm-AMP-1.1a and AC2-WT, respectively. Mutations were introduced to these templates to achieve the desired sequences (shown above).

In the MD simulations, each of the three peptides was placed on top of, but not in direct contact with, a chitin surface that was formed by 14 polymers constructed with the doglycan software using the OPLS-AA force field. Systems were solvated with water using the TIP3P model and then electro-neutralized with NaCl to a final concentration of 150 mM. For each peptide, two replicate simulations were run for 1 μs each. A leap-frog stochastic dynamics integrator was used to integrate Newton's equations of motion with a time-step of 2 fs. Electrostatic interactions were calculated using the PME procedure with a real-space cut-off of 1.2 nm and a Fourier grid spacing of 0.12 nm. Van der Waals interactions were modeled using the classical Lennard-Jones potential with a cut-off of 1.2 nm. The LINCS algorithm was applied to constrain all H-bond lengths. Simulations were run at 1 atm with the Parrinello-Rahman barostat and at 298.15 K with the Berendsen thermostat. Root mean square deviations in structure (RMSD) analyses were performed using GROMACS, with comparisons made against the crystal structure (or initial model) using Ca carbons only.

MM/PBSA analysis for binding free energies. To calculate the peptide-chitin interaction energies, the MM/PBSA method as implemented using the default parameters in the software GMXPBSA 2.1 is used. Briefly, the interaction energy is represented as the sum of the molecular mechanics (MM) energy term and the Poisson-Boltzmann and surface area solvation (PBSA) term. The MM part is calculated as:

E_{MM}=Eᵢₙₜ+E_{coul}+E_{vdw}

Equation 1

MM/PBSA analysis for binding free energies. To calculate the peptide-chitin interaction energies, the MM/PBSA method as implemented using the default parameters in the software GMXPBSA 2.1 is used. Briefly, the interaction energy is represented as the sum of the molecular mechanics (MM) energy term and the Poisson-Boltzmann and surface area solvation (PBSA) term. The MM part is calculated as:

where Eᵢₙₜ involves the bond, angle, and torsions and E_{coul} and E_{vdw} represent the electrostatic and Lennard-Jones energies, respectively. All these terms were extracted using GROMACS. For the PBSA part, the solvation term Gₛₒₗᵥ is composed of polar (Gₚₒₗₐᵣ) and non-polar (Gₙₒₙₚₒₗₐᵣ) energy terms and is calculated as:

Gₛₒₗᵥ=Gₚₒₗₐᵣ+Gₙₒₙ₋ₚₒₗₐᵣ (2)

Equation 2

These terms were calculated using the Adaptive Poisson-Boltzmann Solver (APBS) software. Gₚₒₗₐᵣ corresponds to the energy required to transfer the solute from a low dielectric continuum medium (ε=1) to a continuum medium with the dielectric constant of water (ε=80). In this case, the non-linearized Poisson-Boltzmann equation is used to calculate Gₚₒₗₐᵣ. The Gₙₒₙₚₒₗₐᵣ term is calculated as:

Gₙₒₙ₋ₚₒₗₐᵣ=γSASA+β

Equation 3

where γ=0.0227 kJ·mol⁻¹ Å⁻² and β=0.0 kJ·mol⁻¹. The dielectric boundary was defined using a probe of radius 1.4 Å. This protocol was performed for different “stable states” (see results section), where 100 frames spanning those regions were used for analysis.

Microorganisms and growth media. Escherichia coli DH5α was used as the host for all DNA manipulations and vector storage and was grown in Luria-Bertani medium (LB: 10% tryptone, 5% yeast extract, 5% NaCl) supplemented with 100 μg/mL ampicillin (Amp) when needed. E. coli SHuffle® cells (New England Biolabs, USA) carrying the plasmids of interest were used for recombinant protein expression and were grown using Terrific Broth (TB: 1.2% yeast extract, 2.4% tryptone, 0.5% glycerol, 0.23% KH2PO4, 1.25% K2HPO4) supplemented with 2% glucose and 100 μg/mL Amp. The fungal species Aspergillus niger, Fusarium oxysporum, and Trichoderma reesei were routinely grown on Potato Dextrose Agar (PDA: 0.4% potato peptone, 2% dextrose, 1.5% agar). To obtain spores from the fungal species, PDA plates were seeded with fungal hyphae and grown at 30° C. for one week. Spores were collected from the agar surface with sterile swabs, resuspended in sterile water, quantified by microscopy using a Neubauer chamber, and then stored at 4° C. until use.

DNA manipulation and cloning. Synthetic DNA fragments encoding the peptides AC2-WT, DNv1-AC2, and DNv2-AC2 were obtained from Integrated DNA Technologies (IDT, USA) and then amplified by PCR using primers that annealed at flanking attL sites. PCR products were cloned into the pETG41A plasmid by Gateway™ cloning using the LR Clonase II enzyme mix (Thermo Fisher Scientific, USA) following the manufacturer's instructions, generating the plasmids pETG41A-AC2, pETG41A-DNv1, and pETG41A-DNv2. These vectors encoded the peptides of interest and were fused to maltose-binding protein (MBP) for increased solubility and a His6×tag for affinity purification. Plasmids were electrotransformed into E. coli DH5α. Successful transformants were selected on LB Amp plates; their plasmids were extracted by mini-prep and their constructs were confirmed by PCR and restriction assays. Verified plasmids were then electro-transformed into E. coli SHuffle® cells.

Recombinant Protein Expression and Purification. E. coli SHuffle® cells carrying pETG41A-AC2, pETG41A-DNv1, and pETG41A-DNv2 were grown overnight in TB at 37° C. with agitation. The following morning, 1 L flasks with 500 mL TB were inoculated with a 1:40 dilution of overnight cultures and grown at 37° C. until an OD₆₀₀ of ˜0.4 to 0.6 was reached. IPTG was then added to a final concentration of 0.1 mM, and cells were grown for 16 h at 20° C. Cells were collected by centrifugation, resuspended in lysis buffer (100 mM Tris-HCl, 300 mM NaCl, 5 mM Imidazole, 1 mM PMSF), and disrupted by sonication. Cell debris was removed by centrifugation for 40 min at 8500 rpm and 4° C. The soluble protein fraction was then purified with pre-equilibrated Ni-NTA resin (Qiagen), recovered in elution buffer (100 mM Tris-HCl pH 7.5, 300 mM NaCl, 350 mM Imidazole and 10% glycerol), and dialyzed against protein storage buffer (100 mM TrisHCl pH 7.5, 300 mM NaCl, and 10% glycerol). Protein concentrations were determined using a 96-well based Bradford Assay (Bio-Rad, USA) in a BioTek Multi Plate Reader at A595 nm. Protein purity was checked by SDS-PAGE using SDS-12% polyacrylamide gel and Coomassie blue stain.

Antifungal Activity Assays. To determine the minimum inhibitory concentration (MIC) of peptides in broth dilution assays, fungal spores were inoculated into Yeast Peptone Dextrose medium (YPD, 1% yeast extract, 2% peptone, 2% dextrose) at a concentration of 20,000 spores/ml. The inoculated medium was aliquoted into several tubes, and then serial two-fold dilutions of peptides were prepared. Tubes were incubated at 30° C. for three days and visually inspected for the appearance of hyphal growth. To determine the MIC and IC₅₀ of peptides in agar dilution assays, serial dilutions of antifungal peptides were prepared using protein storage buffer (as described above) and PDA plates were prepared by mixing one volume of tempered agar with one volume of peptide solution. Protein storage buffer was used as a negative control, and 100 μg/mL Zeocin (InvivoGen, USA) was used as positive antifungal control. 20,000 spores were added to the center of the plates before incubating plates at 30° C. for at least 4 days, or until the negative control plate mycelium had grown to half the plate diameter. The diameter of fungal mycelium for each plate was measured and then normalized to the mycelium length of the negative control plate. The resulting data were plotted to interpolate IC₅₀ using an asymmetrical sigmoid curve fit.

### Embedding Model and Similarity Metric

Bi-LSTM Recurrent Neural Networks (RNNs) can learn rich representations for natural language, which enables baseline performance on common tasks. This model architecture learns by examining a sequence of characters in order and trying to predict the next character based on the model's dynamic internal knowledge of the sequences it has seen so far (its “hidden state”). During the training phase, the model gradually revises the way it constructs its hidden state in order to maximize the accuracy of its predictions, resulting in a progressively better statistical summary, or representation, of the protein sequence. To examine what the model learned, the model is interrogated from the amino acid to the proteome level and its internal states are examined. The language model is then fine-tuned using additional tasks, including secondary structure and contact map prediction. In total, the model processed ˜20,000 protein structures in ˜1 day on 2 Nvidia 2080 Ti GPUs.

FIG. 11 shows a representation of training of the models implemented by the system 1 of FIG. 1. 21 million amino acid sequences from PFam and ˜20,000 sequences from the SCOPe database, encoding using amino acid character embeddings, were fed to the model. The model was trained to reconstruct a protein sequence while minimizing cross-entropy loss and then predict information about that sequence such as secondary structure, contact maps, and structural similarity.

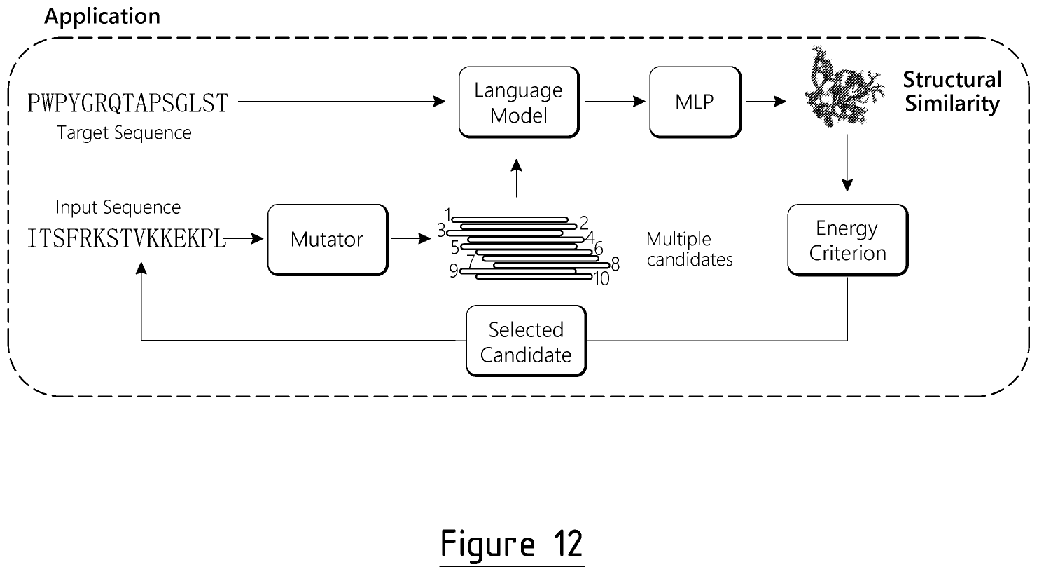

The model used in this work was trained to classify SCOP data (superfamily and fold classes) with 91.12% accuracy this correspond to Module 30 in FIG. 1. Structural similarity between proteins remains challenging to infer solely from amino acid sequences. A previous study developed a method for encoding an amino acid sequence using structural information. Using this method, any protein sequence can be transformed into a vector sequence encoding structural information, with one vector per amino acid position. To assess the accuracy of this approach, the structural similarity scores between proteins is compared to the values obtained by comparing their sequences using this vector-encoding method. The first step in validating the Bi-LSTM model was to validate the structural comparison model. A simple way to approach this problem would be to use metrics such as RMSD or dRMSD for structural comparison; however, these metrics need an equal number of elements to compare. The template modeling (TM) score measures the similarity of two protein structures, is more sensitive to the global fold similarity than to local structural variations, and is length-independent for random structure pairs. Around ˜160,000 protein pairwise comparisons were therefore evaluated based on their TM scores. The results obtained from these pairwise comparisons corresponded with the structural similarity results obtained with the Bi-LSTM model, showing that an increase in the Bi-LSTM model energy criterion also meant a greater structural similarity according to the TM score (FIG. 12). These results show that protein pairs with a structural similarity score (output of Module 30) above 3.5 based on the SCOP hierarchy contain analogous structures, with TM scores ranging from 0.5 to 1.0.

The similarity score between two sequences {i,j} can be defined as a weighted sum of sequential and structural similarities:

score(i,j)=seq(i,j)+str(i,j)

Where the sequential similarity is given by the related probability of an amino acid in a determinate context.

$$P ( x ) = {Π}_{k} P ( {a}_{k} \vert {a}_{1} , {a}_{2} , {a}_{k - 1} )$$

$$seq ( P ( i ) , P ( j ) ) = \sum \qquad P ( i ) log ( \frac{P ( i )}{P ( j )} )$$

Where aₖ is the k-esime amino acid in the sequences, and the comparison scores is a measure of how one probability distribution is different from a second, reference probability distribution.

On the other hand, structural similarity is the probability between amino acids in the sequences to generate similarity in local and global structures.

$${P}_{local} ( x ) = {Π}_{k} P ( {s}_{k} \vert {a}_{1} , {a}_{2} , {a}_{k - 1} )$$

$${P}_{global} ( x ) = {Π}_{\{ i , j \vert i \neq j \}} {P}_{i , j} ( {d}_{i , j} )$$

$${P}_{str} ( x ) = α {P}_{local} ( x ) + β {P}_{global} ( x )$$

$$str ( {p}_{str} , {q}_{str} ) = \sum \qquad p log ( \frac{p}{q} )$$

Where sₖ is the secondary structure of amino acid k-esime, d_{i,j} is the distance in the 3D structure of the amino acid pair {i,j} and α, β are weights to prioritize local or global structure, by default they are equal to 0.5.

FIG. 12 shows a representation of applying the models implemented by the system 1 of FIG. 1. The model was used to generate de novo protein sequences by starting from a random or simple target protein sequence and iteratively mutating it to optimize a certain energy criterion.

De Novo Chitin-Binding Peptides from a Language Model

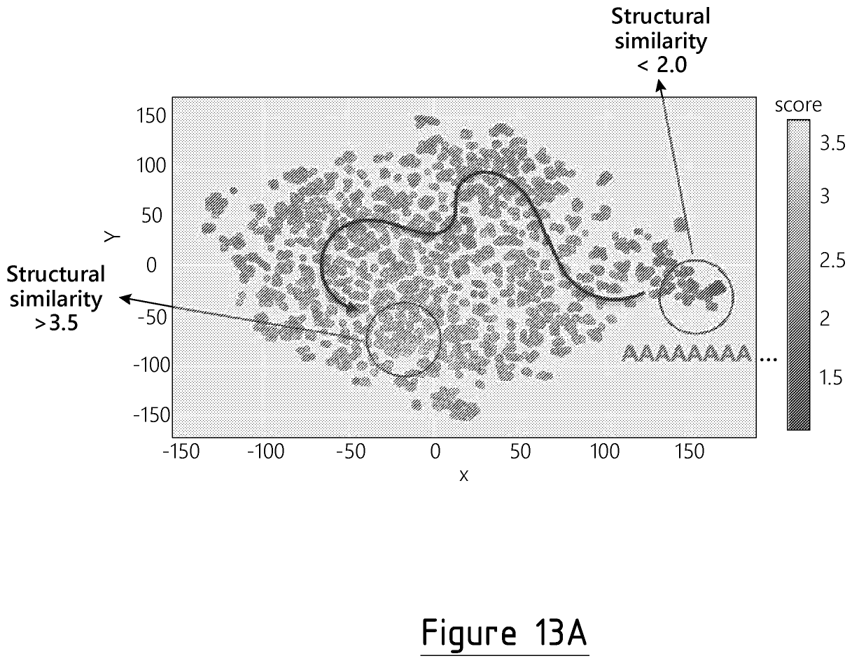

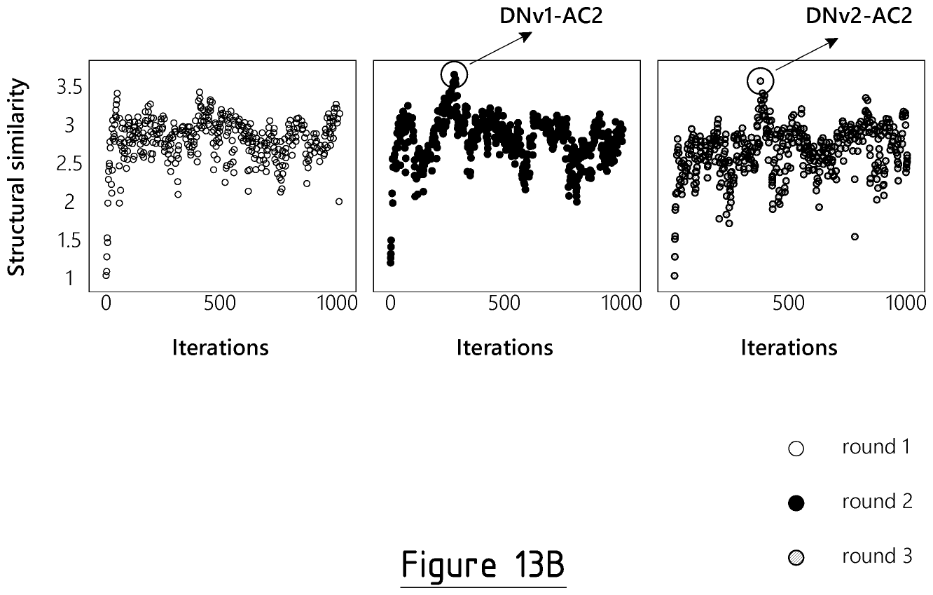

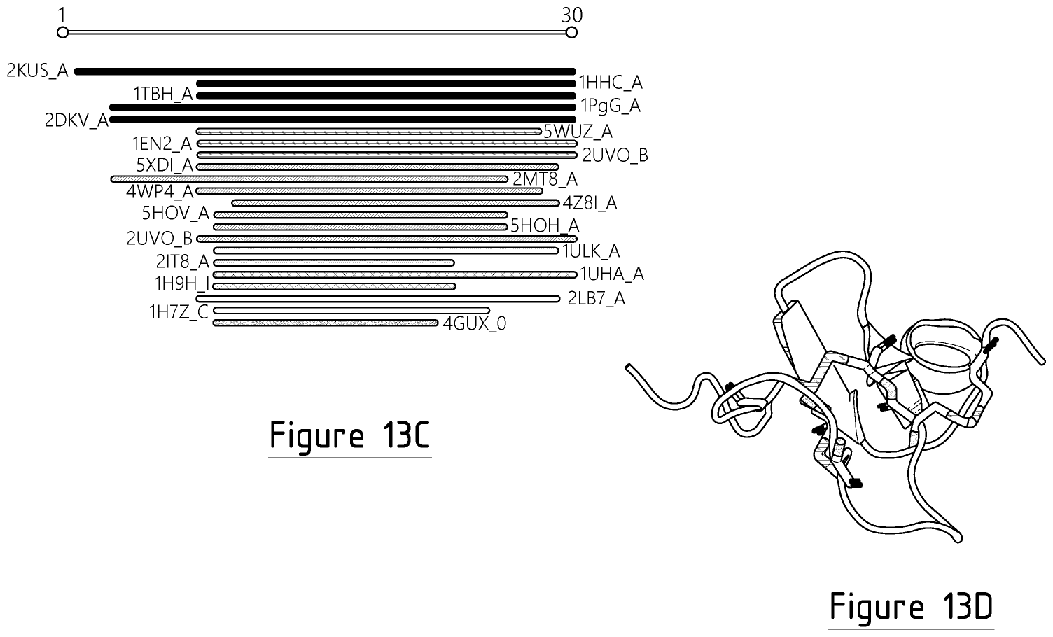

The similarity model searches for analogs of a chitin-binding protein and to design some de novo chitin-binding proteins. Starting from a sequence of poly-alanines, and based on what the language model had learned, the peptide sequence was mutated to obtain a structural similarity score greater than 3.5. This threshold was based on the cut-off selected, for example from FIG. 13B. The mutation process took approximately 4 hours, during which the model explored approximately 20,000 possible sequences (FIG. 13A). The mutation process was performed in three independent rounds, each with a different seed, in order to explore different model trajectories.

Interestingly, the two best de novo matches, DNv1-AC2 and DNv2-AC2, shared just 40% and 54.8% sequence similarity, respectively, with the AC2-WT variant, but were still predicted to have chitin-binding activity. For both of the de novo peptides, the first homologous match obtained by HHpred⁵ was to PDB 2KUS, which corresponds to the antimicrobial peptide Sm-AMP-1.1a, an antifungal peptide with a chitin-binding domain (FIG. 13C). A second match, with a similar score, was to PDB 1MMC, which corresponds to AC2-WT (FIG. 13C). The sequence percentage identity for the de novo peptides DNv1-AC2 and DNv2-AC2 against the 2KUS match was 32.4% and 48.6%, respectively. All of the presented sequence identities do not pass the threshold for structurally reliable homologous alignments, which is determined as a function of the alignment length (which in this case is 30 residues). These peptides could therefore be considered analogous. The two HHpred matches were used for the construction of peptide models (FIG. 13D) and for the following structural analyses of the designed peptide variants.

FIGS. 13A-13D show a representation of design of the de novo protein variants using the system 1 of FIG. 1. (FIG. 13A). Non-dimensional schematization of the de novo protein design pathway, which used the trained language model to implement a series of random mutations in an initial poly-alanine sequence (with an energy criterion below 2.0) in order to generate sequences with an energy criterion above 3.5. (FIG. 13B) Three independent trajectories were run for the de novo peptide design, and the two best candidates (DNv1-AC2 and DNv2-AC2) were selected for analysis. (FIG. 13C) HHPred search results for the peptide variants DNv1-AC2 and DNv2-AC2. (FIG. 13D) Three-dimensional model for DNv1-AC2 built using Modeller, based on the template protein 2KUS.

Molecular Dynamics and Interactions with a Chitin Surface for AC2-WT and the AI-Generated Peptide Variants DNv1-AC2 and DNv2-AC2

1. AC2-WT2

To preliminarily explore the molecular interactions of the tested antifungal peptides (AC2-WT, DNv1-AC2, and DNv2-AC2), unbiased MD simulations of a single free peptide near a chitin surface are performed. Two replicate simulations were run for 1 μs each for each peptide variant. MM/PBSA calculations helped evaluate both the potential for spontaneous binding of the peptides to the chitin surface as well as the strength of the peptide-chitin interaction.

In evaluating these simulations for the AC2-WT antimicrobial peptide, RMSD time-series data showed important deviations between the simulated AC2-WT structure and its crystal structure (FIG. 14). The highest RMSD values that replica 1 achieved were approximately ˜3.0 Å, whereas replica 2 achieved values near −6 Å, when the peptide was compared with its crystal structure (FIG. 14). Both simulations achieved a roughly stable RMSD after the first 50 ns. Interestingly, despite the high RMSD for replica 2, the predicted relative binding energy for replica 2 was either higher (−10.63 to −12.64 kcal·mol⁻¹) than some of the selected structural configurations from replica 1 (−4.36 and −9.11 kcal·mol⁻¹ for configurations 1 and 3 in replica 1, respectively) or within the same order of magnitude (−13.35 kcal·mol⁻¹ for configuration 2 in replica 1) (FIG. 14).

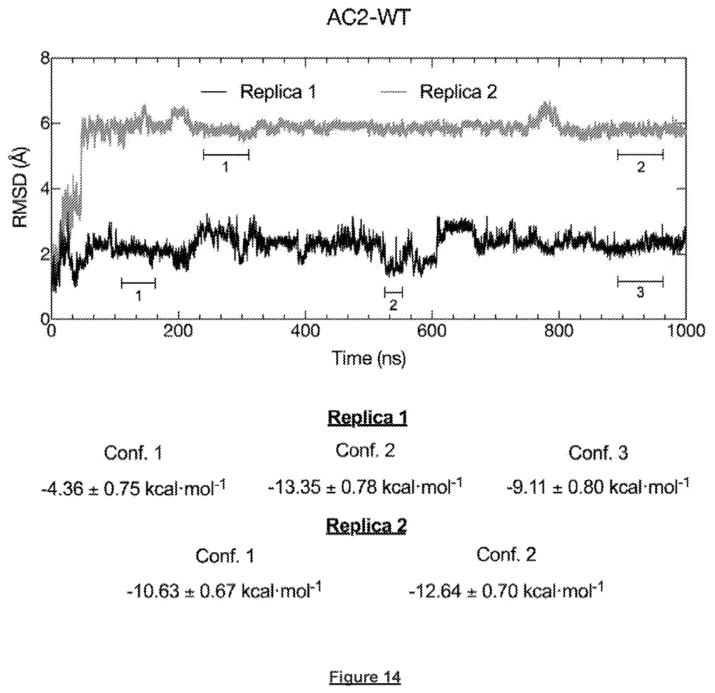

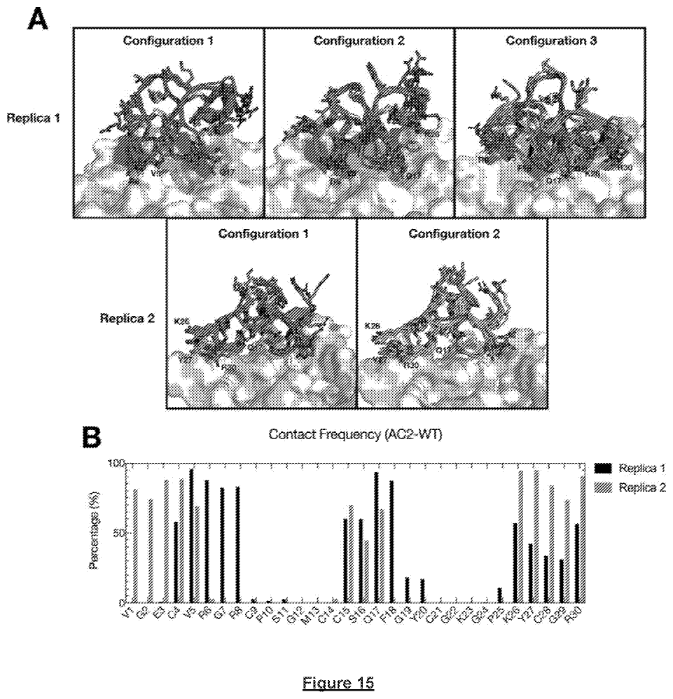

The selected AC2-WT protein configurations with residues that were in contact with the chitin surface are depicted in FIG. 15. In replica 1, the number of interactions increased over time, with more residues seen close to the chitin surface (FIG. 15). However, more peptide-chitin contacts did not translate to higher interaction energy, since the interaction energy changed from −13.35 to −9.11 kcal·mol⁻¹ between configurations 2 and 3 (FIG. 14 and FIG. 15A). In replica 2, most of the peptide-chitin contacts stabilized earlier in the simulations, with no important visual differences (FIG. 15A). Despite the differences among replicas and configurations, there was an observable pattern in the regions in contact with the chitin surface (FIG. 15B), with the beginning, middle, and end of the protein structure involved in most of the contacts. The only difference between replicas was that the initial region of the AC2-WT peptide stayed in contact with the chitin surface in replica 2 but not in replica 1 (FIG. 15B). It is also important to note that AC2-WT interacted with the chitin surface through aromatic residues F18, Y20, and Y27. Aromatic residues have been previously classified as conserved key residue interactions for the hevein-like peptides mechanism.

FIG. 14 shows representations of RMSD and relative binding energies for AC2-WT. RMSD time-series data for AC2-WT across two replicate simulations. Different configurations were obtained at different simulation times, and an MM/PBSA calculation was performed for each configuration. Relative binding free energies are shown at the bottom for each of the configurations depicted in the RMSD plot for each replica.

FIGS. 15A and 15B show representations of contact analyses for AC2-WT. (FIG. 15A) Sample snapshots from the configurations used in the analyses of binding energies for AC2-WT (replicas 1 and 2). Residue numberings are shown for the residues that are visually close to the chitin surface. (FIG. 15B) Per-residue percentage of contact for AC2-WT residues across the entire simulation, for both replica 1 and 2, using a distance cut-off of 4.5 Å.

2. DNV1-AC2

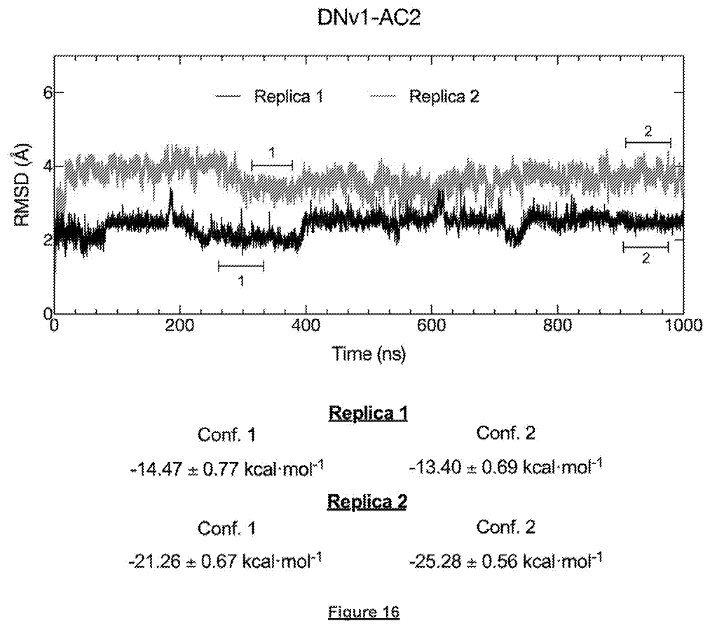

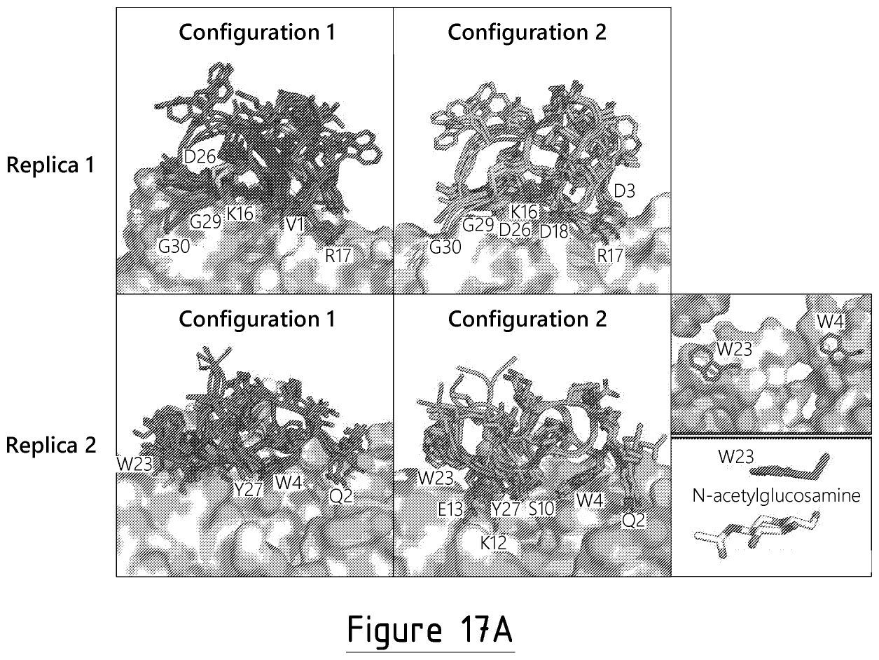

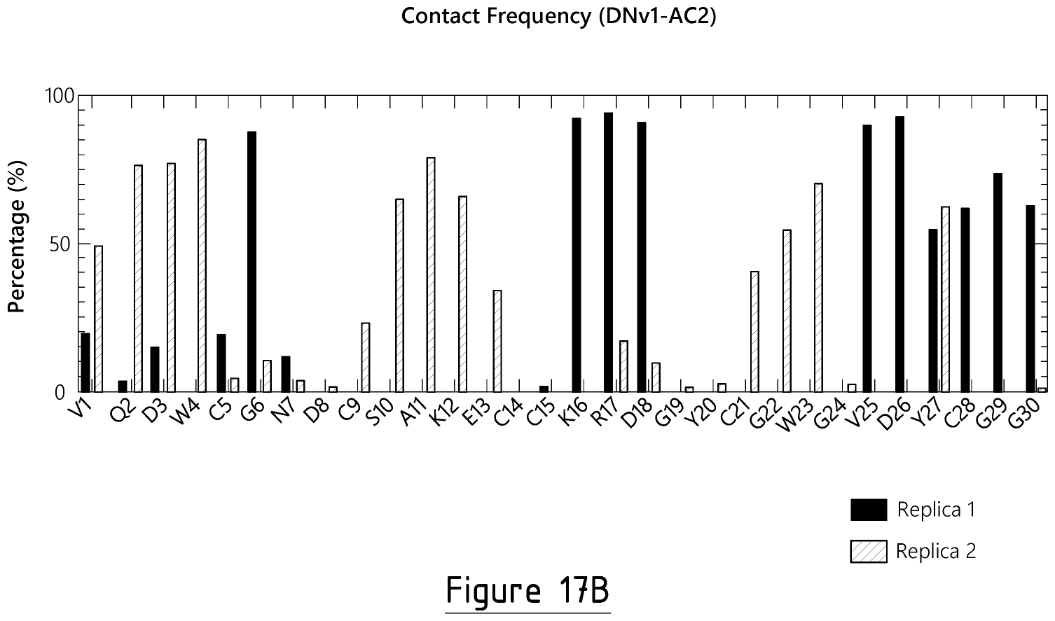

Similar analyses were performed for the AI-generated peptide variant DNv1-AC2. RMSD time-series data showed that the peptide conformation remained close to the initial conformation in both replicas, with average RMSD values of 2.42 and 3.68 Å for replicas 1 and 2, respectively (FIG. 16). These roughly stable conformations were achieved early, after approximately 30 ns of the simulation, which was similar to the AC2-WT simulations (FIG. 16). In terms of binding free energies, the two selected configurations from replica 1 achieved energies of the same magnitude as AC2-WT, with values of −14.47 kcal·mol⁻¹ and −13.40 kcal·mol⁻¹ (FIG. 16). The contact regions in replica 1 of the DNv1-AC2 variant are similar as well, with the main contacts located in the middle and end portions of the structure (FIGS. 17A and 17B) and some contacts locating at the beginning, especially at residue G6, which was in contact with the chitin surface for 83% of the simulation (FIG. 17B).

Interestingly, replica 2 behaved differently from replica 1. The relative binding energies were higher for both selected configurations from replica 2, with values of −21.26 kcal·mol⁻¹ and −25.28 kcal·mol⁻¹ (FIG. 16). The contact area was different from replica 1 as well, with most contacts at the beginning (from residue 1 to 7) and middle (from residue 9 to 13) of the structure (FIGS. 17A and 17B). New contacts were also established in the region between residues 21 and 23 (FIGS. 17A and 17B). One possible explanation for the higher energies of this configuration is the interactions of residue W4 and W23, as shown in FIG. 17A (bottom right panels). These types of interactions, where tryptophan residues are flatly aligned in a CH-π orientation, occur between chitinases and chitin. These interactions are also frequently observed between proteins and sugars. Previous data supports this generalization and suggests that larger aromatic groups have higher association constants and binding enthalpies. In addition, residue E13 can form hydrogen bonds with the amide group of N-acetylglucosamine, which could enhance its ability to bind to the chitin surface.

FIG. 16 shows representations of RMSD and relative binding energies for DNv1-AC2. RMSD time-series data for the DNv1-AC2 peptide variant across two replicate simulations. Different configurations were obtained at different simulation times, and an MM/PBSA calculation was performed for each configuration. Relative binding free energies are shown at the bottom for each of the configurations depicted in the RMSD plot for each replica.

FIGS. 17A and 17B shows representations of contact analyses for DNv1-AC2. (FIG. 17A) Sample snapshots from the configurations used in the analyses of binding energies for the DNv1-AC2 variant (replica 1 and 2). Residue numberings are shown for the residues that are visually close to the chitin surface. (FIG. 17B) Per-residue percentage of contact for DNv1-AC2 residues across the entire simulation, for both replica 1 and 2, using a distance cut-off of 4.5 Å.

3. DNV2-AC2

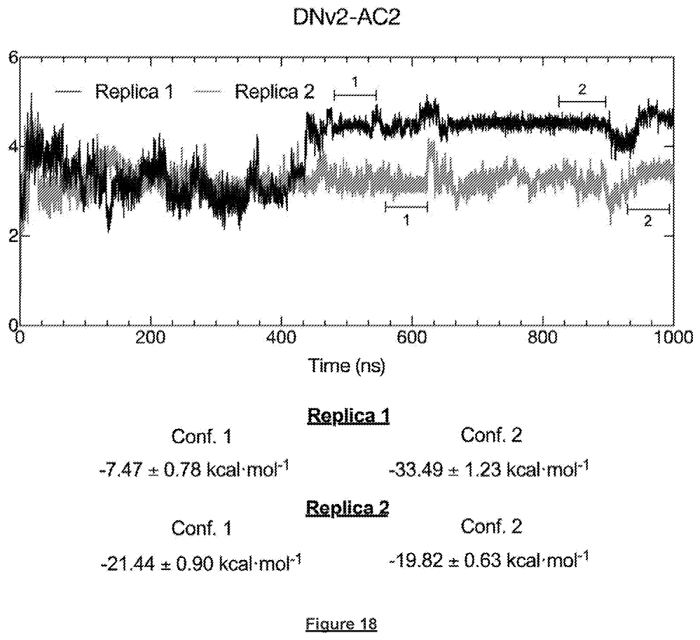

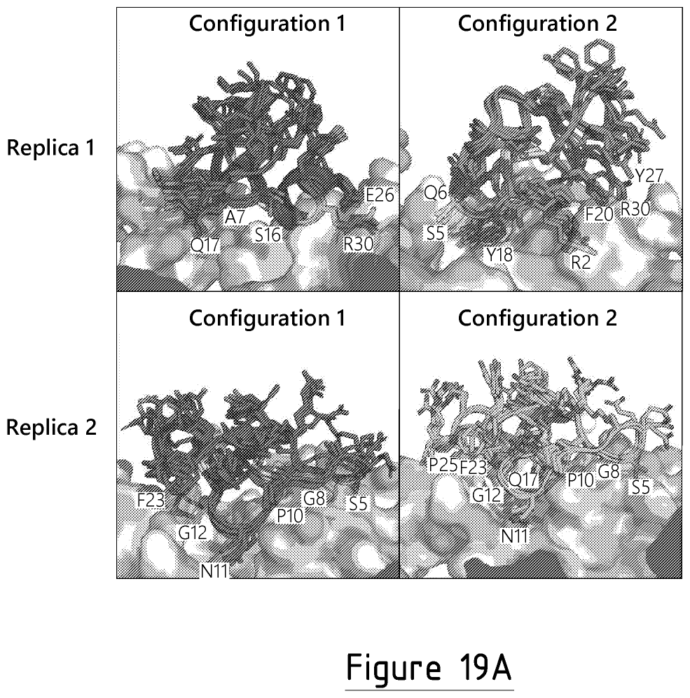

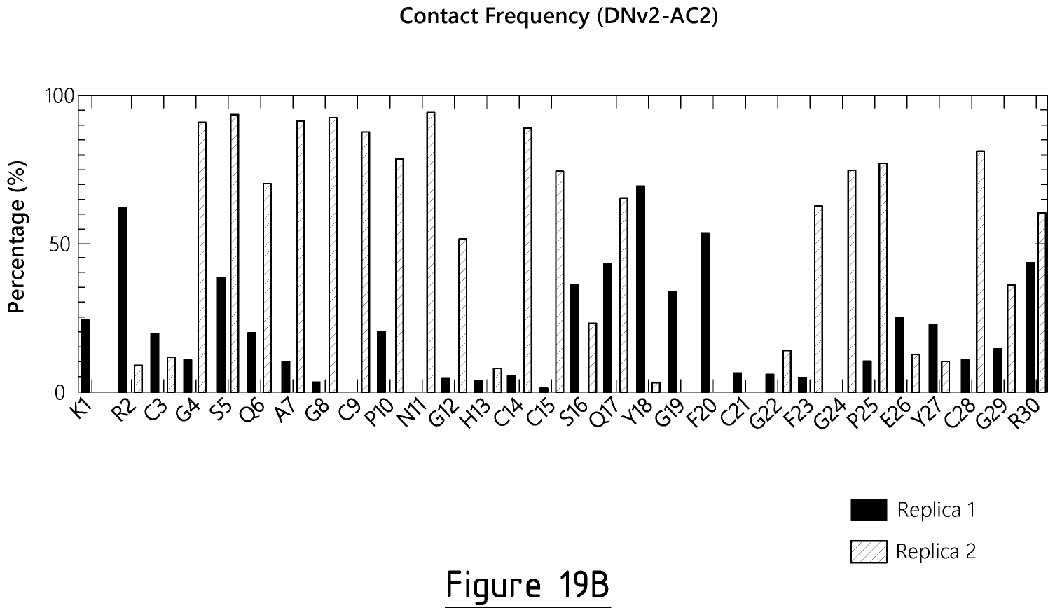

RMSD time-series data for the DNv2-AC2 variant showed similar results to the DNv1-AC2 variant, with values of 3.94 Å and 3.24 Å for replica 1 and 2, respectively (FIG. 18). Two conformations were selected from each replica, spanning the simulation times shown in FIG. 18, and MM/PBSA calculations were performed for each conformation. As shown in FIG. 18, the relative binding energies were dependent on the peptide configuration. For replica 1, the first selected conformation only exhibited an average energy of −7.47 kcal·mol⁻¹ (FIG. 18), whereas the binding energy increased to −33.49 kcal·mol⁻¹ by the second configuration. The main difference between these two configurations is the presence of aromatic residues, such as residues Y18, F20 and Y27, in direct contact with the chitin surface in configuration 2. These residues are similar to the AC2-WT peptide (FIG. 19A), and, as previously mentioned, seem to be important for the peptide's activity.

In replica 2, the peptide-chitin interactions occurred mostly at the hydrophobic residues G4, A7, G8, G12, G24, and G29. Residue F23 was the only aromatic residue in direct contact with the chitin surface (FIG. 19A). These differences may explain the strength of the interaction compared to the second configuration from replica 1 (FIG. 18). However, this peptide-chitin interaction is still stronger than AC2-WT and is similar to the DNv1-AC2 variant, potentially due to residues Q6, Q17, and R30, which can help in the formation of salt bridges and hydrogen bond interactions (FIG. 19). It is important to note that arginine residues, which were present in all the tested variants and were also seen directly interacting with the chitin surface (FIGS. 15, 17, and 19), allow the peptides to interact with anionic components. These interactions also help in the formation of salt bridges and hydrogen bonds Like tryptophan, arginine can participate in cation-7C interactions, which enhances the interactions between peptides and their targets.

FIG. 18 shows representations of RMSD and relative binding energies for DNv2-AC2. RMSD time-series data for the DNv2-AC2 peptide variant across two replicate simulations. Different configurations were obtained at different simulation times, and an MM/PBSA calculation was performed for each configuration. Relative binding free energies are shown at the bottom for each of the configurations depicted in the RMSD plot for each replica.

FIG. 19 shows representations contact analyses for DNv2-AC2. (FIG. 17A) Sample snapshots from the configurations used in the analyses of binding energies for the DNv2-AC2 variant (replicas 1 and 2). Residue numberings are shown for the residues that are visually close to the chitin surface. (FIG. 17B) Per-residue percentage of contact for DNv2-AC2 across the entire simulation, for both replica 1 and 2, using a distance cut-off of 4.5 Å.

### De Novo Peptides Exhibit In Vitro Inhibitory Activity Against Different Fungal Species

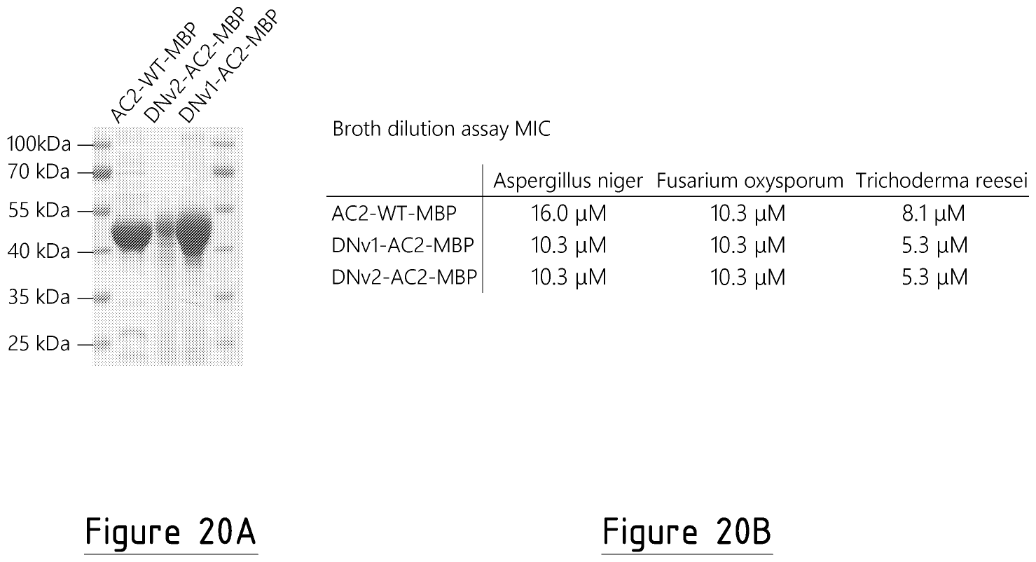

The DNv1-AC2 and DNv2-AC2 de novo peptide designs were validated by testing their antifungal activity and potency relative to the AC2-WT peptide in vitro. DNA encoding the DNv1-AC2, DNv2-AC2, and AC2-WT peptides were cloned into expression vectors to allow recombinant production of the WT peptide and de novo designs in E. coli. All peptides were expressed as chimeric fusions to maltose-binding protein (MBP) in order to improve their solubility and to facilitate purification and downstream handling. All constructs showed a good level of protein production after vector expression was induced and were purified for downstream functional characterization (FIG. 20A).

The activity of the peptide-MBP fusions were tested against three filamentous fungi using a broth dilution assay: Aspergillus niger, Fusarium oxysporum, and Trichoderma reesei (FIG. 20B). AC2-WT-MBP had antifungal activity against all three tested fungi, with MICs ranging from 8 to 16 μM. These results corroborate the phenotype described for the native Ac-AMP2 protein purified from Amaranthus caudatus grains. DNv1-AC2-MBP and DNv2-AC2-MBP both exhibited similar inhibitory activity to AC2-WT when tested against F. oxysporum. Surprisingly, both de novo peptides showed slightly increased activity against A. niger and T. reesei compared to AC2-WT-MBP.

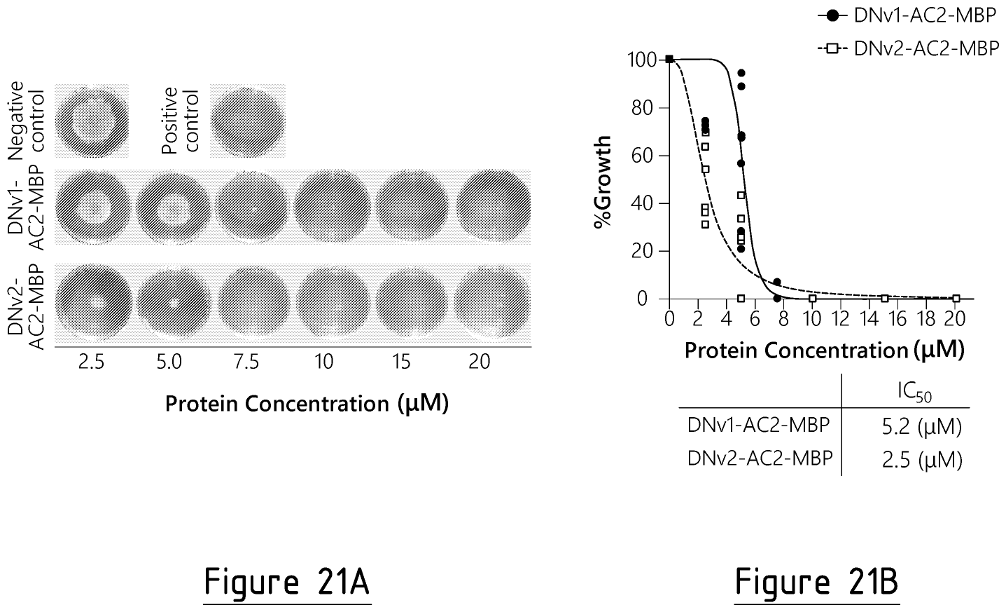

To further investigate the performance of the de novo peptide-MBP fusions, agar dilution assays were used to test for the inhibition of A. niger. The differences in the antifungal potency of both peptides was readily apparent in these assays. As seen in FIG. 21A, DNv1-AC2-MBP slightly decreased mycelium growth at lower concentrations and greatly inhibited growth at concentrations greater than or equal to 7.5 μM. In comparison, DNv2-AC2-MBP negatively impacted mycelium growth even at low micromolar concentrations. The aggregated results of the different experiments and replicates are summarized in FIG. 21B, with IC₅₀ values of 5.2 μM for DNv1-AC2-MBP and 2.5 μM for DNv2-AC2-MBP, indicating a higher potency of the latter peptide variant.

Altogether, these results show that the de novo designed peptides replicated the antifungal activity of the AC2-WT protein, and, in the case of DNv2-AC2, did so with higher potency against the target fungi. These in vitro results confirm the results obtained by molecular dynamics simulations and validate the novel model for de novo protein design.

FIGS. 20A and 20B shows a representation of purification and activity of WT and de novo AC2 peptides fused to MBP protein. (FIG. 20A) SDS-PAGE stained with Coomassie blue dye, showing purified peptide-MBP fusion proteins. (FIG. 20B) Evaluation of the antifungal activity of peptide-MBP fusion proteins. Broth dilution assays were performed to determine the minimum inhibitory concentration (MIC) of each peptide against three fungal species. Each peptide-MBP dilution in broth media was tested in duplicate, and growth was assessed visually.

FIGS. 21A and 21B shows a representation of Agar dilution assay comparing the antifungal activity of DNv1-AC2-MBP and DNv2-AC2-MBP against A. niger. (FIG. 21A) Representative images of agar dilution assays performed with serial dilutions of de novo peptide-MBP fusions added to agar before solidification. Assays tested for the inhibition of mycelial growth from A. niger spores. Protein storage buffer was used as a negative control for growth inhibition, and Zeocin was used as positive control. (FIG. 21B) IC₅₀ values for DNv1-AC2-MBP and DNv2-AC2-MBP. Aggregated results from three independent experiments were used to interpolate IC₅₀ from the fitted curve.

### DISCUSSION AND CONCLUSIONS

In this work, a new deep-learning model (for example, the system 1) is developed, trained, and shown how it can be used to guide and improve protein design. Specifically, module 20 can design analog proteins based on a representation learning architecture that contains evolutionary and structural information from millions of protein sequences. Given that the study of sequence-structure relationships in proteins is a highly dimensional task, the system described in module 20 (FIG. 1) can play an important role in this area and complement similar models. The system 1 including module 20 (FIG. 1) produced high-quality predictions, with an accuracy of 91.12% when classifying structural information from the SCOP database. These results are similar to other models available in the literature. Additionally, it is shown that it is possible to generate de novo protein sequences with a particular function. This is demonstrated by generating peptides with antifungal activity. Interestingly, the model can generalize, as shown by its ability to successfully generate functional proteins starting from a simple poly-alanine sequence. To put the capabilities of the model in context, the study of the predicted de novo antifungal peptides by running molecular dynamics simulations, evaluating the interactions involved in the process of chitin recognition, and observing patterns such as the importance of aromatic residues to the strength the binding interactions.

To date, numerous methods have been described for generating novel antifungal peptide designs. Directed and rational approaches have focused on comparing the sequences and structures of antifungal peptides to identify the key elements that impact antifungal potency, such as peptide cationicity and hydrophobicity, peptide tertiary structure and the distribution of the residues within that structure, and peptide length and amphipathicity. On the other hand, machine learning methods have become an attractive alternative for predicting peptide sequences with antifungal activity; different publications have explored this approach by using and combining a variety of approaches like support vector machines, hidden Markov models (HMM) and character embedding. The system described in module 20 (FIG. 1) generated de novo peptide sequences with potent in vitro antifungal activity that was comparable to wild-type antifungal variants such as AC2-WT. In the case of DNv2-AC2, the de novo peptide even showed improved potency compared to its native counterpart, highlighting the power of the approach to generate novel and useful peptide variants.

The results presented in this work open a huge door for the development of new alternative proteins and peptides, and the model presented here has potential applications in industries such as food manufacturing, agriculture, cosmetics, and therapeutics, as well as in the design of new proteins with specific activities. By providing an example of the artificial generation of functional protein analogs, this manuscript broaches an important topic and encourages discussions of simplicity and limits in nature, especially in terms of the possible structural arrangements a protein can adopt. Homology is the classical definition for similarity at the sequence, structure and function levels, but no clear definition exists for considerable structural similarities despite low sequence identities. The term “remote homology” has been used to describe similar structures with a sequence identity of 25% or lower; this type of homology is usually inferred from common features, such as functional residues, or from unusual structural features. On the other hand, the term “analogy” usually refers to two or more proteins with no common origin that converge to similar structural features. Both cases are difficult to experimentally validate and their definitions have changed throughout the years. Finally, the system 1 incorporating the module 20 (FIG. 1) has certain advantages over the state-of-art models, such as its low complexity (i.e., fewer parameters) and its inclusion of structural information. Limitations of the model are related to the size variability of the generated embeddings, which makes it difficult for comparison and interpretability.

In general, the word “module,” as used herein, refers to logic embodied in hardware or firmware, or to a collection of software instructions, possibly having entry and exit points, written in a programming language, such as, for example, Java, Lua, C or C++. A software module may be compiled and linked into an executable program, installed in a dynamic link library, or may be written in an interpreted programming language such as, for example, BASIC, Perl, or Python. It will be appreciated that software modules may be callable from other modules or from themselves, and/or may be invoked in response to detected events or interrupts. Software modules configured for execution on computing devices may be provided on a computer readable medium, such as a compact disc, digital video disc, flash drive, or any other tangible medium. Such software code may be stored, partially or fully, on a memory device of the executing computing device, such as the processing system 1200, for execution by the computing device. Software instructions may be embedded in firmware, such as an EPROM. It will be further appreciated that hardware modules may be comprised of connected logic units, such as gates and flip-flops, and/or may be comprised of programmable units, such as programmable gate arrays or processors. The modules described herein are preferably implemented as software modules. They may be represented in hardware or firmware. Generally, the modules described herein refer to logical modules that may be combined with other modules or divided into sub-modules despite their physical organization or storage.

A model may generally refer to a machine learning construct which may be used to automatically generate a result or outcome. A model may be trained. Training a model generally refers to an automated machine learning process to generate the model that accepts an input and provides a result or outcome as an output. A model may be represented as a data structure that identifies, for a given value, one or more correlated values. For example, a data structure may include data indicating one or more categories. In such implementations, the model may be indexed to provide efficient look up and retrieval of category values. In other embodiments, a model may be developed based on statistical or mathematical properties and/or definitions implemented in executable code without necessarily employing machine learning.

Machine learning generally refers to automated processes by which received data is analyzed to generate and/or update one or more models. Machine learning may include artificial intelligence such as neural networks, genetic algorithms, clustering, or the like. Machine learning may be performed using a training set of data. The training data may be used to generate the model that best characterizes a feature of interest using the training data. In some implementations, the class of features may be identified before training. In such instances, the model may be trained to provide outputs most closely resembling the target class of features. In some implementations, no prior knowledge may be available for training the data. In such instances, the model may discover new relationships for the provided training data. Such relationships may include similarities between proteins such as protein functions.

Each of the processes, methods, and algorithms described in the preceding sections may be embodied in, and fully or partially automated by, code modules executed by one or more computer systems or computer processors comprising computer hardware. The code modules may be stored on any type of non-transitory computer-readable medium or computer storage device, such as hard drives, solid state memory, optical disc, and/or the like. The systems and modules may also be transmitted as generated data signals (for example, as part of a carrier wave or other analog or digital propagated signal) on a variety of computer-readable transmission mediums, including wireless-based and wired/cable-based mediums, and may take a variety of forms (for example, as part of a single or multiplexed analog signal, or as multiple discrete digital packets or frames). The processes and algorithms may be implemented partially or wholly in application-specific circuitry. The results of the disclosed processes and process steps may be stored, persistently or otherwise, in any type of non-transitory computer storage such as, for example, volatile or non-volatile storage.

The various features and processes described above may be used independently of one another, or may be combined in various ways. All possible combinations and sub-combinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Any process descriptions, elements, or blocks in the flow diagrams described herein and/or depicted in the attached figures should be understood as potentially representing modules, segments, or portions of code which include one or more executable instructions for implementing specific logical functions or steps in the process. Alternate implementations are included within the scope of the embodiments described herein in which elements or functions may be deleted, executed out of order from that shown or discussed, including substantially concurrently or in reverse order, depending on the functionality involved, as would be understood by those skilled in the art.

All of the methods and processes described above may be embodied in, and partially or fully automated via, software code modules executed by one or more specially configured general purpose computers. For example, the methods described herein may be performed by a processing system, card reader, point of sale device, acquisition server, card issuer server, and/or any other suitable computing device. The methods may be executed on the computing devices in response to execution of software instructions or other executable code read from a tangible computer readable medium. A tangible computer readable medium is a data storage device that can store data that is readable by a computer system. Examples of computer readable mediums include read-only memory, random-access memory, other volatile or non-volatile memory devices, compact disk read-only memories (CD-ROMs), magnetic tape, flash drives, and optical data storage devices.

It should be emphasized that many variations and modifications may be made to the above-described embodiments, the elements of which are to be understood as being among other acceptable examples. All such modifications and variations are intended to be included herein within the scope of this disclosure. The foregoing description details certain embodiments. It will be appreciated that no matter how detailed the foregoing appears in text, the systems and methods can be practiced in many ways. As is also stated above, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

Further detail regarding embodiments relating to the systems and methods disclosed herein, as well as other embodiments, is provided in the Appendix of the present application, the entirety of which is bodily incorporated herein and the entirety of which is also incorporated by reference herein and made a part of this specification. The Appendix provides examples of features that may be provided by a system that implements at least some of the functionality described herein, according to some embodiments, as well as specific system configuration and implementation details according to certain embodiments of the present disclosure.

What is claimed is:

1. A method, comprising:
receiving a first input comprising a target protein sequence having a feature of interest;
receiving a second input comprising a candidate protein;
applying a first machine learning model to the target protein sequence to generate fragments of interest from the target protein sequence;
applying the first machine learning model to the candidate protein to generate fragments from the candidate protein that corresponds to the fragments of interest from the target protein sequence;
applying a second machine learning model to the fragments of interest and the fragments from the candidate protein, wherein applying the second machine learning model comprises generating an encoded representation in a multidimensional space for each of the fragments of interest and the fragments from the candidate protein;
generating a similarity score between the target protein sequence and the candidate protein based on a similarity between the fragments of interest from the target protein sequence and the fragments from the candidate protein;
generating a hierarchical scale of similarity between the target protein sequence and a plurality of candidate proteins comprising the candidate protein according to the feature of interest and the similarity score; and
selecting candidate proteins from the plurality of candidate proteins based on the hierarchical scale, wherein higher scores on the hierarchical scale indicate candidate proteins or the more similar substitute candidate proteins inputs for each of the interest inputs.

2. The method of claim 1, wherein the first machine learning model is configured to generate the fragments based on splitting the target protein sequence and the candidate protein into functional domain fragments.

3. The method of any of claims 1 and 2, wherein the second machine learning module comprises a plurality of fully-connected layers.

4. The method of any of claims 1-3, wherein the second machine learning module comprises a plurality of convolutional neural network layers.

5. The method of any of claims 1-4, wherein the second machine learning module comprises a plurality of recurrent neural network layers configured to identify the beginning and end of functional domains.

6. The method of any of claims 3-5, where the layers are connected with direct connections or residual neural networks connections.

7. The method of claim 1, wherein the computational algorithm to split comprises a module to allow the input to be divided into a defined size.

8. The method of claim 1, wherein the first machine learning model is configured to generate the fragments of interest and the fragments from the candidate protein with different sizes or functional domains.

9. The method of claim 1, wherein applying the second machine learning model comprises encoding an amino-acid representation of the candidate protein in a multidimensional space given a local context of the candidate protein.

10. The method of claim 9, wherein encoding the amino-acid representation comprises applying a sub-model having a plurality of fully-connected layers, a plurality of convolutional neural networks layers, or a plurality of recurrent neural networks layer to compress the amino-acid representation given the local context of the candidate proteins.

11. The method of claim 10, where the layers are connected with direct connections or residual neural networks connections.

12. The method of claim 9, further comprising training the sub-model to predict a probability of the amino acid beginning or ending at a certain position in the candidate protein given the local context.

13. The method of claim 9, further comprising training the sub-model using all known or predicted protein sequences.

14. The method of any of claims 1-13, wherein the second machine learning model comprises a sub-model to encode the candidate proteins and the target protein in a multidimensional space given a protein sequence and a compress positional representation information.

15. The method of claim 14, wherein the sub-model comprise a plurality of fully-connected layers, a plurality of convolutional neural networks layers, or a plurality of recurrent neural networks layer to compress the amino-acid representation given the local context of the candidate proteins.

16. The method of claim 15, where the layers are connected with direct connections or residual neural networks connections.

17. The method of claim 14, further comprising training the sub-model to predict a protein structure, and a function of protein, and a contact-map of protein;

18. The method of any of claims 1-17, wherein generating a similarity score between the target protein sequence and each candidate protein comprises applying a third machine learning model to generate the similarity score between the target protein and the one or more candidate proteins.

19. The method of claim 18, wherein the third machine learning model comprises a plurality of fully-connected layers, a plurality of convolutional neural networks layers, or a plurality of recurrent neural networks layer to compare two multidimensional protein representations.

20. The method of claim 18, wherein the similarity score is a hierarchical number that contains information of the protein sequence, structure and function.

21. The method of 20, further comprising training the third machine learning to predict similarity of proteins using the SCOP hierarchical information.

22. The method of claim 1, where the module ranker orders the candidates from the best rated to the worst.

23. A method of use a computational system implemented by one or more computers to:
receiving a plurality of inputs, each input is a protein sequence where one or more are animal protein and the rest of the inputs are plant-based, animal-free candidate proteins;
processing each of the plurality of plant-based, animal-free candidate proteins inputs with a computational algorithm to split the protein in fragments of interest;
processing each of the plurality of inputs with an artificial intelligence model to get an encoded representation in a multidimensional space;
processing each of the plurality of inputs to generate a similarity score between the animal protein inputs and the plant-based, animal-free candidate proteins;
generating a hierarchical scale of similarity between animal protein inputs and plant-based, animal-free candidate proteins; and
selecting the higher scores or the more similar substitute candidate proteins inputs for each of the interest inputs.

24. The method of use of claim 23, wherein the plant-based, animal-free candidate proteins are split into functional domains and/or into a defined size.

25. The method of use of any of claims 23 and 24, wherein the plant-based, animal-free candidate proteins and the animal-origin protein are encoded using a trained artificial intelligence model, given an encoded representation of proteins and/or fragments.

26. The method of use of any of claims 23-25, wherein the plant-based, animal-free candidate proteins previously encoded in a multidimensional space are compared using a hierarchical similarity metric.

27. The method of use of any of claims 23-26, wherein the plant-based, animal-free candidate proteins previously compared using a hierarchical similarity metric are ranked from the best rated to the worst.

28. The method of use of claim 20, wherein the best plant-based, animal-free candidate proteins are selected given a number of max candidates or if the score exceeds a threshold.

29. The method of use of claim 20, wherein the best selected plant-based, animal-free candidate proteins are bioinformatically simulated and/or synthesized in the laboratory to verify the activity and fulfill the desired function.

30. A system comprising a processor and a memory including instruction to program the processor to perform the method of any of claims 1-29.

31. The method of any of claims 1-29, wherein the at least one fragment of interest is a target antifungal protein.

32. The method of any of claims 1-29, further comprising identifying, based on substitute candidate proteins, alternative antifungal proteins to a target antifungal protein, wherein the at least one fragment of interest comprises a feature of the target antifungal protein.

33. The method of any of claims 1-29, further comprising identifying, based on substitute candidate proteins, alternative enzymes to a target enzyme, wherein the at least one fragment of interest comprises a feature of the target enzyme.


SEQUENCE LISTING (5 sequences)

SEQ ID NO: 1 - protein, 30 aa, Artificial Sequence
  AC2
VGECVRGRCPSGMCCSQFGYCGKGPKYCGR

SEQ ID NO: 2 - protein, 30 aa, Artificial Sequence
  DNv1-AC2
VQDWCGNDCSAKECCKRDGYCGWGVDYCGG

SEQ ID NO: 3 - protein, 30 aa, Artificial Sequence
  DNv2-AC2
KRCGSQAGCPNGHCCSQYGFCGFGPEYCGR

SEQ ID NO: 4 - protein, 194 aa, Artificial Sequence
  Protein 1
MGSIPIAIMSALAMVYLGAKDSTRTQINKVVRFDKLTKPNDVYSFSLASRLYAEERYPILPEYLQCVKSQTNGIIRNVLQPSSVDSQTAMVLVNAIVFKGMYQIGLFRVASMASEKMKILELPFASGTWTSSNVMEERKIKVYLPRMKMEEKYNLTSVLMQAVHAAHAEINEAGREVVGSAEAGVDAASRCVSP

SEQ ID NO: 5 - protein, 208 aa, Artificial Sequence
  Protein 2
ESISNQTDVALSMTKLLLSKEARDKLLSFLRSKSTDYLNSFASQLVTLLPSFKHVVNSDYKANLASVDFQTKAVEVASEVFANALYFKGAWNEKFDASLTKDHDFHLLGGNSRYKQGEDKRQFSMYFFLPDAKDGLPALAEKLASFGFEASDVLKELGVVLPFTVGGLTEMVESPVGQTIMLRSARFSSKIDFVADHPFLFLIKEDLT